(19) United States
(12) Patent Application Publication
(10) Pub. No.: US 2025/0388696 A1
(43) Pub. Date: Dec. 25, 2025

(54) FUSION PROTEINS WITH HALF LIFE EXTENDING DOMAINS

(71) Applicant: Navigo Proteins GmbH, Halle/Saale (DE)

(72) Inventors: Ulrich Haupts, Halle/Saale (DE); Eva Bosse-Doenecke, Halle/Saale (DE); Gregor Hoffmann, Halle/Saale (DE); Manja Gloser-Braeunig, Halle/Saale (DE)

(73) Assignee: Navigo Proteins GmbH, Halle/Saale (DE)

(21) Appl. No.: 18/880,225

(22) PCT Filed: Jun. 30, 2023
(86) PCT No.: PCT/EP2023/068109

§ 371 Date: Dec. 30, 2024

(30) Foreign Application Priority Data
Jun. 30, 2022 (EP) 22182401.4
Nov. 4, 2022 (EP) 22205669.9

(51) Int. Cl. C07K 16/32; C07K 14/435; C07K 16/40

(52) U.S. Cl. CPC C07K 16/32; C07K 14/435; C07K 16/40; C07K 2317/92; C07K 2317/94; C07K 2319/31

(57) ABSTRACT

The invention relates to a fusion protein that comprises at least one albumin binding moiety and further comprises a tumor targeting moiety. The invention relates to the use of the fusion proteins or of compositions comprising the fusion proteins for medical applications.

### FIELD OF THE INVENTION

The invention relates to a fusion protein that comprises at least one albumin binding moiety and further comprises a tumor targeting moiety. The invention relates to the use of the fusion proteins or of compositions comprising the fusion proteins for medical applications.

### BACKGROUND OF THE INVENTION

The present invention provides novel fusion proteins binding with affinity for serum albumin and a tumor target. The fusion proteins of the invention are particularly well-suited for medical applications with tailored half-life of specific targets to tumor related proteins.

The above overview does not necessarily describe all problems solved by the present invention.

### SUMMARY OF THE INVENTION

The present disclosure provides the following items 1 to 12, without being specifically limited thereto:

This summary of the invention is not limiting, and other aspects and embodiments of the invention will become evident from the following description, examples and drawings.

1. A fusion protein comprising
(a) a binding protein for serum albumin comprising the amino acid sequence of SEQ ID NO: 33, or an amino acid sequence with 1 or 2 substitutions, deletions, or insertions to SEQ ID NO: 33, and having a binding affinity to serum albumin of less than 25 nM, and
(b) a targeting moiety having a binding affinity of less than 100 nM, preferably less than 10 nM, to a protein expressed by tumors.

2. The fusion protein according to item 1, wherein the binding protein for serum albumin comprises an amino acid sequence selected from the group of SEQ ID NO: 1-32, or comprises an amino acid sequence with 1 or 2 substitutions, deletions, or insertions to SEQ ID NO: 1-32, and has a binding affinity to serum albumin of less than 25 nM.

3. The fusion protein according to item 1 or 2, comprising a binding protein for human serum albumin or mouse serum albumin.

4. The fusion protein according to any one of items 1-3, wherein the targeting moiety is a non-immunoglobulin protein, preferably a ubiquitin mutein, a mutein of domains of protein A, ankyrin repeat protein mutein, a lipocalin mutein, a mutein of human Fyn SH3 domain, a mutein of the tenth domain of human fibronectin, a mutein of FN3 domain, a mutein of Kunitz domains, a Sac7d mutein, a chagasin mutein, a mutein of multimerized low density lipoprotein receptor-A, a mutein of cysteine-knot miniprotein, a mutein of Stefin, a mutein of Armadillo-repeat protein, a mutein of tetranectin, a mutein of C-type lectin domain, or a mutein of CTLA-4, or wherein the targeting moiety is an immunoglobulin, an immunoglobulin fragment or variant thereof, a single domain antibody, or single chain variable fragment (scFv) of an antibody.

5. The fusion protein according to item 4, wherein the non-Immunoglobulin protein comprises an ubiquitin mutein exhibiting 80% to 94% identity to ubiquitin (SEQ ID NO: 43).

6. The fusion protein according to any one of items 1-5, wherein the targeting moiety binds to a protein expressed by tumors such as Her2, FAP, or ED-B.

7. The fusion protein according to any one of items 1-6, for use in diagnostics or in the treatment of cancer, wherein the half-life (serum or blood) of the targeting moiety of the fusion protein is longer than the half-life (serum or blood) of the targeting moiety without the serum albumin binding protein.

8. A nucleic acid molecule encoding the fusion protein according to any one of items 1-6.

9. A vector comprising the nucleic acid molecule of item 8.

10. A host cell or a non-human host comprising the fusion protein as defined in any one of items 1 to 6, a nucleic acid as defined in item 8, and/or a vector of item 9.

11. A composition comprising the fusion protein as defined in any one of items 1-6, a nucleic acid as defined in item 8, and/or a vector of item 9.

12. A method for the production of the fusion protein as defined in any one of items 1-6, comprising culturing of the host cell of item 10 under conditions suitable to obtain said fusion protein, and optionally isolating said fusion protein.

### BRIEF DESCRIPTION OF THE FIGURES

FIG. 1. Affinity of fusion proteins comprising albumin binding domains and targeting moieties to human serum albumin and target (FIG. 1A: extradomain B of fibronectin (ED-B), FIG. 1B: human epidermal growth factor receptor 2 (Her2), FIG. 1C: fibroblast activation protein (FAP)). The K_{D} for each fusion protein was determined at pH 7.3 by SPR. All fusion proteins bind with K_{D} of less than 25 nM to human serum albumin and bind with K_{D} of less than 10 nM to the specific target.

FIG. 2. Stability of fusion proteins comprising albumin binding domains and targeting moieties to human serum albumin in serum. Shown are FAP specific targeting moieties fused to the albumin binding domain of d01 (SEQ ID NO: 9; as dimer), FIG. 2A shows the stability of the fusion protein 218702 in serum. FIG. 2B shows the stability of fusion protein 218690 in serum after prolonged incubation. The affinity of the fusion protein to the target is not significantly reduced after 24 h incubation in serum.

FIG. 3: Biodistribution of a fusion protein comprising a Her2 specific targeting moiety and albumin binding domains shows very high tumor specific accumulation and excellent tumor/blood ratios.

The fusion protein 218464 comprising a Her2-specific targeting moiety and SEQ ID NO: 9 (d01) and the fusion protein 218462 comprising a Her2-specific targeting moiety and SEQ ID NO: 6 (c20) were radiolabeled with 177Lu to a specific activity of ˜ 1 MBq/nmol and injected IV to mice (SKOV-3 xenograft model). The in vivo biodistribution was evaluated at 5 terminal time points over 168 hours (1 mouse/time point). The in vivo study analyzed the level of the fusion protein in blood, tumor, kidneys and liver. The fusion proteins show excellent tumor accumulation because of longer half-life of the fusion protein compared to ubiquitin mutein without albumin binding domain. The half-life of the fusion protein is about 36 h. The albumin binding domain of the fusion protein results in higher tumor accumulation due to long half-life and excellent tumor/blood ratio. Further, low accumulation in kidneys and liver was observed.

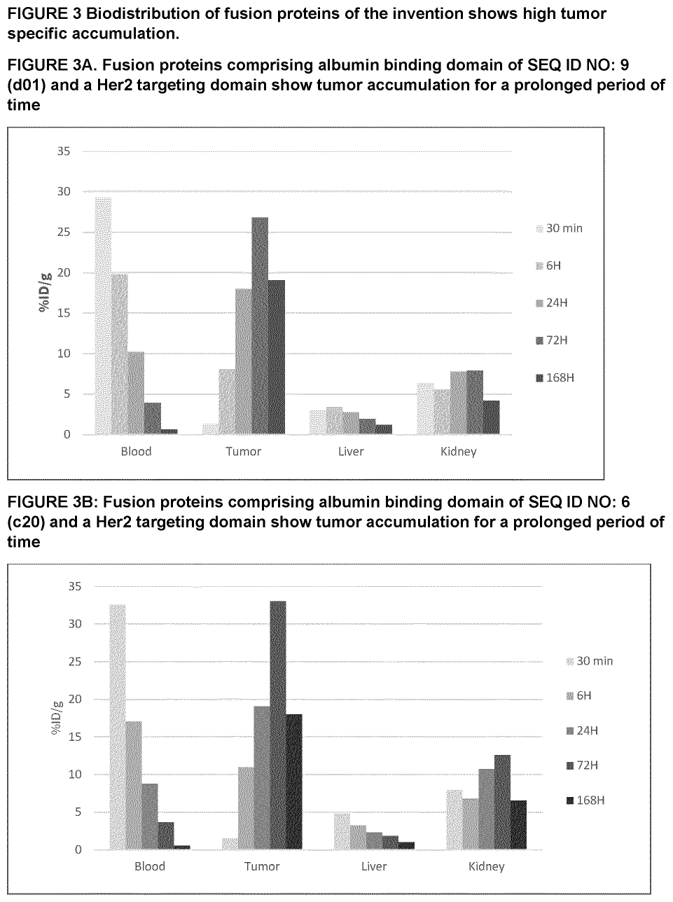

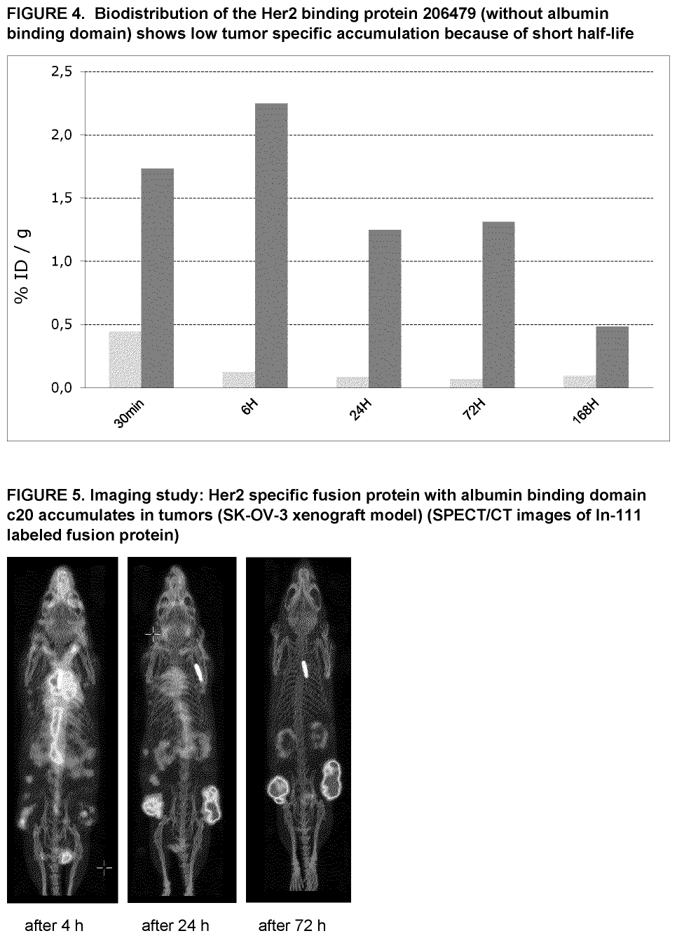

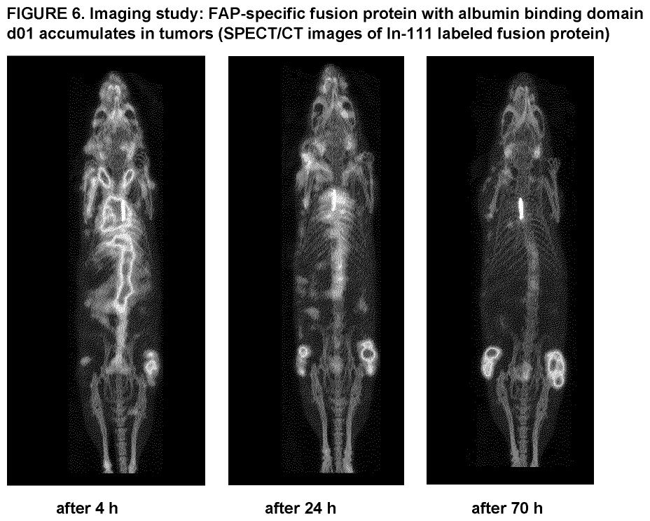

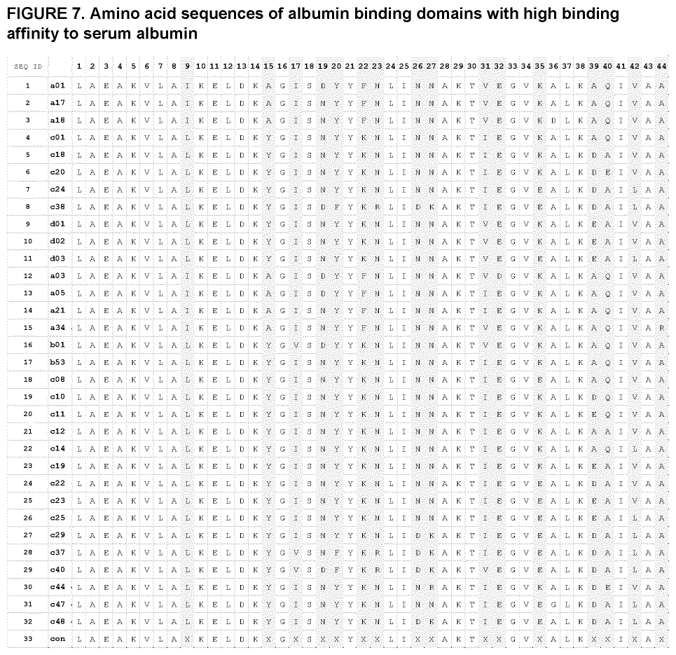

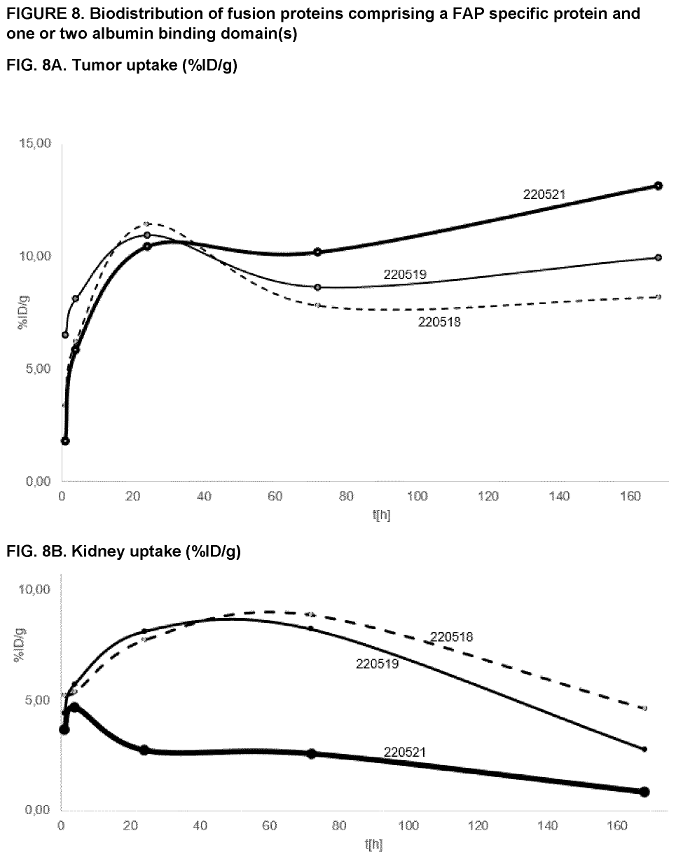

FIG. 3A: The fusion protein comprising the albumin binding domain of SEQ ID NO: 9 (d01) fused to a Her2-specific ubiquitin mutein show excellent tumor accumulation for a prolonged period of time.

FIG. 3B: The fusion protein comprising the albumin binding domain of SEQ ID NO: 6 (c20) fused to an Her2-specific binding moiety show excellent tumor accumulation for a prolonged period of time.

FIG. 4: Biodistribution of Her2 specific targeting moiety shows low tumor specific accumulation because of short half-life. For experimental settings, see FIG. 3. Light grey columns refer to wildtype ubiquitin, dark grey columns refer to refers to the ubiquitin mutein as targeting moiety with binding specificity for the cancer target Her2. The half-life (in blood) of the cancer specific targeting moiety is very short (i.e. without fusion to the albumin binding domain as disclosed herein).

FIG. 5: Imaging study: fusion protein 218462 (206479_(G4S) 4_c20) accumulates in tumors (SK-OV-3 xenograft model) after 72 h. Shown are SPECT/CT images 4 h, 24 h, and 72 h after dosing with In-111 labeled compound 1 (˜30 MBq/mouse), maximal intensity projection (MIP) images. Uptake is presented as percent injected dose per gram tissue (% ID/g). The Her2 specific fusion protein clearly accumulates in tumors after 3 days due to the fusion with the albumin binding domain.

FIG. 6: Imaging study: FAP-specific fusion protein 220521 with albumin binding domain d01; SEQ ID NO: 47) accumulates in tumors (hFAP overexpressing HEK293 cell xenograft model) after 24 and 70 h. Shown are SPECT/CT images 4 h, 24 h, and 70 h after dosing with In-111 labeled compound 1 (˜21 MBq/mouse), maximal intensity projection (MIP) images. Uptake is presented as percent injected dose per gram tissue (% ID/g). The FAP specific fusion protein clearly accumulates in tumors after a prolonged time due to the fusion with the albumin binding domain.

FIG. 7: Amino acid sequences of albumin binding domains with high binding affinity to HSA. High binding affinity refers to a binding affinity of a K_{D} of maximum 25 nM.

FIG. 8: Biodistribution of a fusion protein comprising a FAP specific targeting moiety and one or two albumin binding domain(s) shows very high tumor specific accumulation. Fusion proteins 220518 (SEQ ID NO: 55), 220519 (SEQ ID NO: 54), 220521 (SEQ ID NO: 47) comprising a FAP-specific targeting moiety (SEQ ID NO: 42) and SEQ ID NO: 9 (d01) were radiolabeled with ¹⁷⁷Lu and ˜20 MBq/nmol at a dose of 100 nmol/kg were and injected IV to female NMRI nude mice (CHO-hFAP overexpression clone; xenograft model). The in vivo study analyzed the level of the fusion protein in tumor and kidney at different time points for 168 h. 220518: thick dashed line; 220519: thin line; 220521: thick line.

FIG. 8A: The fusion proteins show increased tumor accumulation (% ID/g) for a prolonged period of time.

FIG. 8B: The fusion proteins efficiently reduce the kidney uptake (% ID/g).

### DETAILED DESCRIPTION OF THE INVENTION

The present inventors have developed a solution to meet the strong ongoing need in the art for expanding medical options for the diagnosis and treatment of cancer by providing novel fusion proteins with high affinity to serum albumin and to a cancer target. The fusion protein comprises at least one domain that binds to (human or mouse) serum albumin. Further, the fusion proteins as defined herein are functionally characterized by specific affinity for human serum albumin and for a cancer target (such as Her2, ED-B, or FAP). Due to the fusion of the tumor specific targeting domain to the human serum albumin binding domain, the half-life of the binding protein for a tumor target is extended. In particular, the invention provides fusion proteins that comprise at least one ubiquitin mutein (also known as Affilin® molecule) and a serum albumin binding domain as described herein.

Such fusion proteins may broaden so far unmet medical strategies for the diagnosis and therapy of cancer. In particular, the fusion proteins may be used for diagnostic or imaging purposes, for example, for the presence of tumor cells expressing the target of the targeting moiety, and for radiotherapy treatment of tumors expressing the target. The new proteins are engineered to enable all relevant steps for medical use.

Before the present invention is described in detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art to which this invention belongs.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland). 5 Several documents (for example: patents, patent applications, scientific publications, manufacturer's specifications, instructions, etc.) are cited throughout the text of this specification. Nothing herein is to be construed as an admission that the invention is not entitled to antedate such disclosure by virtue of prior invention. Some of the documents cited herein are characterized 10 as being “incorporated by reference”. In the event of a conflict between the definitions or teachings of such incorporated references and definitions or teachings recited in the present specification, the text of the present specification takes precedence.

All sequences referred to herein are disclosed in the accompanying sequence listing (WIPO ST.26 compliant xml.-file) that, with its whole content and disclosure, forms part of the disclosure content of the present specification.

### General Definitions of Important Terms Used in the Application

Throughout this specification and the appended claims, unless the context requires otherwise, the word “comprise”, and variations such as “comprises” and “comprising”, will be understood to imply the inclusion of a stated integer or step or group of integers or steps but not the exclusion of any other integer or step or group of integers or steps. The term “comprise(s)” or “comprising” may encompass a limitation to “consists of” or “consisting of”, should such a limitation be necessary for any reason and to any extent.

The term “about”, as used herein, encompasses the explicitly recited amounts as well as deviations therefrom of up to +20%. More preferably, a deviation of up to +15%, more preferably of up to +10%, and most preferably up to 5% is encompassed by the term “about”. The term “at least about 10, 20, 30, 40, 50, 60, 70, 80 amino acid residues” is not limited to the concise number of amino acid residues but also comprises amino acid stretches that comprise up to 20% additional or comprise up to 20% less residues.

The term “fusion protein” relates to a protein comprising at least a first amino acid chain joined genetically to at least a second amino acid chain. Thus, a fusion protein may comprise a multimer of proteins/peptides which are expressed as a single, linear polypeptide. It may comprise one, two, three, four, or even more proteins/peptides. For example, a fusion protein can be created through joining of two or more genes that originally coded for separate proteins/peptides. As will be explained below in greater detail, the “fusion protein” of the invention comprises at least two components, namely (i) at least one target binding moiety and (ii) at least one serum albumin binding moiety.

The term “moiety” or “domain” refers to a sub-structure which is part of a protein or fusion protein. The terms “binding protein” and “binding domain” may be used interchangeably herein, e.g. the terms “serum albumin binding protein” and “serum albumin binding domain” may be used interchangeably herein.

The term “fused” means that the components are linked by peptide bonds, either directly or via peptide linkers.

A “linker” as used herein is an amino acid sequence that joins at least two moieties. The linker as understood herein is a peptide linker of up to 30 amino acids. Two or more moieties may be linked via a peptide linker of 5, 6, 7, 8, 9, 10, 11, 12, 13, 14, 15, 16, 17, 18, 19, 20, 21, 22, 23, 24, 25, 26, 27, 28, 29, or 30 amino acids. A linker may be comprised of amino acids such as glycine and serine and may be glycine-rich (e.g., more than 50% of the residues in the linker can be glycine residues). However, other linkers for the fusion of proteins are known in the art and can be used herein. In the fusion protein of the present invention, the serum albumin binding domain and the tumor-targeting moiety may be operably linked by a linker, in particular a peptide linker. The term “operably linked” as used herein refers to a positioning of the components such that they function in their intended manner. The fusion protein of the present invention may comprise one or more serum albumin binding domains, and/or may comprise one or more tumor targeting moieties. In various embodiments, the fusion protein comprises at least two tumor targeting moieties, which may be located at the C-terminus or at the N-terminus of the fusion protein. In various other embodiments, the fusion protein comprises, or consist of, at least two serum albumin binding domains and at least two tumor targeting moieties. The one or more serum albumin binding domains, and/or the one or more tumor-targeting moieties may be operably linked by a linker, in particular a peptide linker. Also, a linker, in particular a peptide linker, may be present between the one or more serum albumin binding domains and the one or more tumor targeting moieties.

Such a linker is operably linking the respective terminally located serum albumin binding domain and tumor-targeting moiety. In various embodiments, the fusion protein comprises at least two serum albumin binding domains, which may be located at the C-terminus or at the N-terminus of the fusion protein. In various embodiments, the fusion protein comprises, or consists of, at least two serum albumin binding domains and (no more than) one tumor-targeting moiety (or tumor-specific binding protein). The at least two serum albumin binding domains may be operably linked by a linker as described above. Likewise, the at least two tumor-targeting moieties (or tumor-specific binding proteins) may be operably linked by a linker as described above. In various preferred embodiments, a linker as described above may be a peptide linker, more specifically a Gly-Ser linker. In preferred embodiments, the linker may be a G4S or a (G4S)2 linker, preferably a (G4S)2 linker. Accordingly, in preferred embodiments, the linker may have an amino acid sequence selected from GGGGSGGGGS (SEQ ID NO: 48), GGGGSGGGGSGGGGSGGGGS (SEQ ID NO: 49), and GGGGSGGGGSGGGGS (SEQ ID NO: 50).

The terms “protein” and “polypeptide” refer to any chain of two or more amino acids linked by peptide bonds, and does not refer to a specific length of the product. Thus, peptides, proteins, amino acid chain, or any other term used to refer to a chain of two or more amino acids, are included within the definition of “polypeptide”, and the term polypeptide may be used instead of, or interchangeably with, any of these terms. The term polypeptide is also intended to refer to the products of post-translational modifications of the polypeptide, which are well known in the art. The term “albumin” as used herein refers to “serum albumin”, more specifically human serum albumin (HSA) or mouse serum albumin (MSA). HSA is the most abundant protein found in the blood. The terms “binding protein/domain for albumin” and “binding protein/domain for serum albumin” may be used interchangeably herein. Further, as used herein, the term “binding protein/domain for serum albumin” is directed to an (isolated) non-natural binding domain for albumin, in particular an (isolated) non-natural binding domain for serum albumin. The term “non-natural” as used herein refers to a protein or domain that is synthetic, i.e., having an amino acid sequence not present in native polypeptides. The term “(serum) albumin binding domain” as used herein refers to a peptide or polypeptide that may bind albumin in vivo and/or in vitro. The albumin binding domain of the fusion protein of the present invention may bind (serum) albumin with an affinity of less than 25 nM in vivo and/or in vitro. In the present invention, albumin (serum albumin) may be derived from any animal species, for example, human, monkey, or rodent, preferably human.

As used herein, the targeting moiety having a binding affinity of less than 100 nM to a protein expressed by tumors may be considered as a tumor targeting moiety (or tumor protein targeting moiety). A tumor-targeting moiety as used herein is directed to an (isolated) non-natural targeting moiety having binding affinity for a protein expressed by tumors. Further, a tumor-targeting moiety as used herein refers to a peptide or polypeptide that may bind a tumor protein (i.e., a protein expressed by tumors) in vivo and/or in vitro. The tumor-targeting moiety of the fusion protein of the present invention may bind to a tumor protein with an affinity of less than 100 nM in vivo and/or in vitro. In the present invention, tumor proteins may be derived from any animal species, for example, human, monkey, or rodent, preferably human.

In various embodiments, the tumor-targeting moiety of the fusion protein of the present invention is a therapeutically effective tumor-targeting moiety, i.e. effective for treatment of cancer. In various other embodiments, the tumor-targeting moiety of the fusion protein of the present invention is a diagnostically effective tumor-targeting moiety, i.e. effective for diagnosing cancer. The terms “tumor” and “cancer” may be used interchangeably herein. Likewise, the terms “tumor cell” and “cancer cell” may be used interchangeably herein. The terms “tumor” and “cancer” as used herein refer to or describe the physiological condition in mammals, preferably humans, in which a population of cells are characterized by unregulated cell growth. Examples of cancer include, but are not limited to, carcinoma, blastoma, sarcoma, and hematologic cancers such as lymphoma and leukemia. The term “tumor” as used herein refers to any mass of tissue that results from excessive cell growth or proliferation, either benign (noncancerous) or malignant (cancerous) including pre-cancerous lesions. Tumor growth is generally uncontrolled and progressive, does not induce or inhibit the proliferation of normal cells.

In various embodiments of the present invention, the tumor-targeting moiety binds to a ligand of an immune checkpoint protein expressed on cancer cells, e.g. PD-L1 and PD-L2. In various embodiments of the present invention, the tumor-targeting moiety binds to the extracellular domain of a protein expressed by a tumor (or tumor cells). In various embodiments of the present invention, the protein expressed by tumor cells is a protein whose expression is upregulated in tumors (or tumor cells). In various embodiments of the present invention, the protein expressed by tumor cells is the extracellular domain of a protein whose expression is upregulated in tumors (or tumor cells). In various embodiments of the present invention, the protein expressed by a tumor (or tumor cells) is a tumor antigen or a tumor-associated/specific antigen expressed by a tumor (or tumor cells). Tumor antigens or a tumor-associated/specific antigens are typically overexpressed antigens (i.e., antigens whose expression is upregulated). In preferred embodiments of the present invention, the tumor-targeting moiety binds to a ligand selected from any one of prostate specific membrane antigen (PSMA), folate receptor FOLR1 (folate receptor alpha) and FOLR2, extra domain B of oncofetal human fibronectin (ED-B), and epidermal growth factor receptor (EGFR).

The term “Her2” refers to human epidermal growth factor receptor 2; synonym names are ErbB-2, Neu, CD340 or p185). Human Her2 is represented by the NCBI accession number NP_004439; the extracellular domain (residues 1-652) of Her2 is represented by the UniProt Accession Number P04626. The term “Her2” comprises all polypeptides which show a sequence identity of at least 70%, 80%, 85%, 90%, 95%, 96% or 97% or more, or 100% to NP_004439 and have the functionality of Her2. Her2 is overexpressed in several tumor types, for example, in 15-30% of all mamma carcinoma.

The term “FAP” as used herein refers to Fibroblast Activation Protein (FAP) which is also known as prolyl endopeptidase FAP, dipeptidyl peptidase FAP, integral membrane serine protease, surface-expressed protease, etc. The term “FAP” refers to UniProt accession number Q12884 and all polypeptides which show a sequence identity of at least 70%, 75%, 80%, 85%, 90%, 95%, 96% or 97% or more, or 100% to the FAP of UniProt accession number Q12884 (human) and have the functionality of FAP. The human FAP is 89.5% identical to mouse FAP (accession number P97321), 88.6% identical to rat FAP and 99.6% identical to cynomolgus FAP (accession number A0A2K5VGF4). The term “FAP” includes the extracellular domain (residues 26-760). For example, FAP is expressed in epithelial tumors and malignant sarcoma cells. The expression of FAP was found in activated stromal fibroblasts of more than 90% of all human carcinomas.

The terms “ED-B” or “EDB” refer to extra domain B of oncofetal human fibronectin (Uniprot accession number P02751-7). ED-B is an extracellular matrix oncoprotein, that is expressed by newly formed blood vessels in (solid) tumors, lymphoma and in some leukaemia. ED-B occurs in an oncofetal fibronectin isoform between two fibronectin domains Fn^{III}7 and Fn^{III}8.

The term “Affilin” or “Affilin®” (registered trademark of Navigo Proteins GmbH) as used herein refers to binding proteins based on ubiquitin muteins. The term “Affilin” as used herein refers to derivatives of ubiquitin which differ from ubiquitin or from proteins with at least 80%, and up to and including 94%, amino acid identity to ubiquitin (SEQ ID NO: 43) by amino acid exchanges, insertions, deletions, or any combination thereof and a specific binding affinity to a target.

The term “binding” according to the invention preferably relates to a specific binding to a protein target, such as serum albumin or a tumor specific target protein.

The term “dissociation constant” or “K_{D}” defines the specific binding affinity. As used herein, the term “K_{D}” (usually measured in “nanomol/L”, sometimes abbreviated as “nM”) is intended to refer to the dissociation equilibrium constant of the particular interaction between a binding protein (e.g., Her2 specific Affilin) and a target protein (e.g. Her2). As disclosed herein, a binding affinity of a serum albumin binding protein of the present invention to serum albumin of less than 25 nM means that a serum albumin binding protein of the present invention binds to serum albumin with a K_{D} of less than 25 nM. As described elsewhere herein, the present invention encompasses serum albumin binding proteins having a (moderate) binding affinity to serum albumin of 25 nM or more, in particular between 25 nM and 3 μM. Such serum albumin binding proteins bind to serum albumin with a K_{D} of 25 nM or more, in particular with a K_{D} between 25 nM and 3 μM.

As further disclosed herein, a binding affinity of a tumor targeting moiety of the present invention to a protein expressed by tumors of less than 100 nM means that a tumor targeting moiety of the present invention binds to a protein expressed by tumors with a K_{D} of less than 100 nM.

The term “modification” or “amino acid modification” refers to a substitution, a deletion, or an insertion of a reference amino acid at a particular position in a parent/reference polypeptide sequence by another amino acid. Given the known genetic code, and recombinant and synthetic DNA techniques, the skilled person can readily construct DNAs encoding the amino acid variants. As used herein, “substitutions” are defined as exchanges of an amino acid by another amino acid. Given the known genetic code, and recombinant and synthetic DNA techniques, the skilled person can readily construct DNAs encoding the amino acid variants. The term “insertions” comprises the addition of amino acid residues to the original amino acid sequence wherein the original amino acid sequence remains stable without significant structural change. The term “deletion” means that one or more amino acid residues are taken out of the original sequence and the amino acids originally N-terminal and C-terminal of the deleted amino acid are now directly connected and form a continuous amino acid sequence.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein. Methods for alignment are well known in the art. For example, the SIM Local similarity program is preferably employed (Xiaoquin Huang and Webb Miller (1991), Advances in Applied Mathematics, vol. 12: 337-357), that is freely available. For multiple alignment analysis, ClustalW is preferably used (Thompson et al. (1994) Nucleic Acids Res., 22 (22): 4673-4680).

Each amino acid of the query sequence that differs from the reference amino acid sequence at a given position is counted as one difference. An insertion or deletion in the query sequence is also counted as one difference. The sum of differences is then related to the length of the reference sequence to yield a percentage of non-identity.

### Embodiments of the Invention

The present invention will now be further described in more detail. Each embodiment defined below may be combined with any other embodiment or embodiments unless clearly indicated to the contrary. In particular, any feature indicated as being preferred or advantageous may be combined with any other feature or features indicated as being preferred or advantageous.

Fusion Proteins that Bind to HSA with High Affinity.

The present invention relates to a fusion protein comprising a binding protein for albumin wherein the albumin binding protein comprises the amino acid sequence of SEQ ID NO: 33, or an amino acid sequence with 1 or 2 substitutions, deletions, or insertions thereto, and a targeting moiety selected from a non-Immunoglobulin protein or antibody or antibody fragment that is capable of binding to a tumor specific targeting protein. The present invention relates to a fusion protein comprising a binding protein for albumin wherein the albumin binding protein comprises the amino acid sequence with at least 95% sequence identity to SEQ ID NO: 33, and a targeting moiety selected from a non-Immunoglobulin protein or antibody or antibody fragment that is capable of binding to a tumor specific targeting protein.

The binding protein for albumin comprises the amino acid sequence of SEQ ID NO: 33, or it comprises an amino acid sequence with 1 or 2 substitutions, deletions, or insertions in the amino acid sequence of SEQ ID NO: 33, wherein SEQ ID NO: 33 has an amino acid sequence as shown herein:

LAEAKVLAX₉KELDKX₁₅GX₁₇SX₁₉X₂₀YX₂₂X₂₃LIX₂₆X₂₇AKTX₃₁X₃₂GV
X₃₅ALKX₃₉X₄₀IX₄₂AX₄₄,

wherein X₉ is L or I, X₁₅ is Y or A, X₁₇ is I or V, X₁₉ is N or D, X₂₀ is Y or F, X₂₂ is K or F, X₂₃ is N or R, X₂₆ is N or D, X₂₇ is N, K, or R, X₃₁ is I or V, X₃₂ is E or D, X₃₅ is K or E, X₃₉ is D, A, or E, X₄₀ is E, A or Q, X₄₂ is V or L, and/or X₄₄ is A or R.

L=Leucine (Leu), A=Alanine (Ala), E=Glutamic acid (Glu), K=Lysine (Lys), V=Valine (Val), D=Aspartic acid (Asp), G=Glycine (Gly), S=Serine (Ser), Y=Tyrosine (Tyr), I=Isoleucine (Ile), T=Threonine (Thr), N=Asparagine (Asn), F=Phenylalanine (Phe), R=Arginine (Arg), Q=Glutamine (Gln), P=Proline (Pro), M=Methionine (Met), W=Tryptophan (W), H=Histidine (His), C=Cysteine (C).

In preferred embodiments, the fusion protein comprises an albumin binding protein with an amino acid sequence with 1 or 2 substitutions, deletions, or insertions of SEQ ID NO: 33 that has in position 20 an aromatic amino acid such as Y or F. In preferred embodiments, the amino acid sequence with 1 or 2 substitutions, deletions, or insertions of SEQ ID NO: 33 has not a G, L, or D in position 20 wherein the K_{D} to human HSA is below 25 nM.

In preferred embodiments, the fusion protein comprises an albumin binding protein with an amino acid sequence with 1 or 2 substitutions, deletions, or insertions of SEQ ID NO: 33 that has an aromatic amino acid such as Y in position 21. In some embodiments, the amino acid sequence with 1 or 2 substitutions, deletions, or insertions of SEQ ID NO: 33 has not a G in position 21 wherein the K_{D} to human HSA is below 25 nM.

In preferred embodiments, the fusion protein comprises an albumin binding protein with an amino acid sequence with 1 or 2 substitutions, deletions, or insertions of SEQ ID NO: 33 that has a G in position 33. In some embodiments, the amino acid sequence with 1 or 2 substitutions, deletions, or insertions of SEQ ID NO: 33 has not an S, L, or V in position 33 wherein the K_{D} to human HSA is below 25 nM.

In preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein the 1 or 2 substitutions, deletions or insertions are made at positions other than positions 9, 15, 17, 19, 20, 22, 23, 26, 27, 31, 32, 35, 39, 40, 42, and 44 of SEQ ID NO: 33. Accordingly, in such embodiments, the substitutions in positions 9, 15, 17, 19, 20, 22, 23, 26, 27, 31, 32, 35, 39, 40, 42, and 44 according to SEQ ID NO: 33 remain unaffected. In other preferred embodiments, the 1 or 2 substitutions, deletions or insertions in the amino acid sequence of SEQ ID NO: 33 are made at any one of positions 20, 21 and/or 33 of SEQ ID NO: 33, as described herein above. Accordingly, in such embodiments, the substitutions in positions 9, 15, 17, 19, 22, 23, 26, 27, 31, 32, 35, 39, 40, 42, and 44 according to SEQ ID NO: 33 remain unaffected.

In various preferred embodiments, the 1 or 2 substitutions, deletions, or insertions in the amino acid sequence of SEQ ID NO: 33 are 1 or 2 substitutions or deletions in the amino acid sequence of SEQ ID NO: 33, more preferably 1 or 2 substitutions in the amino acid sequence of SEQ ID NO: 33.

In preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₃₁ is I, X₃₂ is E, X₃₉ is D, and X₄₀ is E (as shown for SEQ ID NO: 6).

In preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₃₁ is V, X₃₂ is E, X₃₉ is E, X₄₀ is A (as shown for SEQ ID NO: 9).

In further preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₉ is L, and more preferably the amino acids of positions 1-14 consist of amino acids LAEAKVLALKELDK (SEQ ID NO: 51). In other further preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₉ is I, and more preferably the amino acids of positions 1-14 consist of amino acids LAEAKVLAIKELDK (SEQ ID NO: 52).

In some embodiments, the fusion protein comprises an albumin binding protein with an amino acid sequence with 1 or 2 substitutions, deletions, or insertions of SEQ ID NO: 33 wherein the amino acid in positions 1, 2, 3, 4, 5, 6, 7, 8, 10, 11, 12, 13, 14, 16, 18, 21, 24, 25, 28, 29, 30, 33, 34, 36, 37, 38, 41, 43 are not substituted or deleted. In such embodiments, the 1 or 2 substitutions, deletions, or insertions in the amino acid sequence of SEQ ID NO: 33 are made at 1 or 2 positions selected from positions 9, 15, 17, 19, 20, 22, 23, 26, 27, 31, 32, 35, 39, 40, 42, and 44 of SEQ ID NO: 33, wherein in case of “1 or 2 substitutions”, these differ from the substitutions defined for these positions in SEQ ID NO: 33. In preferred embodiments, the “1 or 2 substitutions” include a substitution at position 20 of SEQ ID NO: 33, wherein such a substitution preferably still provides for an aromatic amino acid, as described herein above.

In some embodiments, the binding protein for albumin comprises an amino acid sequence with at least 95% sequence identity to the amino acid sequence of SEQ ID NO: 33 depicted above. The 95% sequence identity allows for up to two modifications, in particular substitutions, in the amino acid sequence of SEQ ID NO: 33 at positions other than positions 9, 15, 17, 19, 20, 22, 23, 26, 27, 31, 32, 35, 39, 40, 42, and 44. Accordingly, in such embodiments, the substitutions in positions 9, 15, 17, 19, 20, 22, 23, 26, 27, 31, 32, 35, 39, 40, 42, and 44 according to SEQ ID NO: 33 remain unaffected. In various preferred embodiments, the binding protein for albumin comprises an amino acid sequence with at least 95% sequence identity to the amino acid sequence of SEQ ID NO: 33, wherein the 95% sequence identity allows for up to two modifications, in particular substitutions, in the amino acid sequence of SEQ ID NO: 33 at any of positions 20, 21, and/or 33 of SEQ ID NO: 33. Accordingly, in such embodiments, the substitutions in positions 9, 15, 17, 19, 22, 23, 26, 27, 31, 32, 35, 39, 40, 42, and 44 according to SEQ ID NO: 33 remain unaffected.

The present invention provides a fusion protein comprising a binding protein for albumin wherein the albumin binding protein comprises the amino acid sequence of SEQ ID NO: 1-32, or an amino acid sequence with 1 or 2 substitutions, deletions, or insertions thereto wherein the K_{D} to HSA is below 25 nM, and a targeting moiety selected from a non-Immunoglobulin protein or antibody or antibody fragment that is capable of binding to a tumor specific targeting protein.

The present invention provides a fusion protein comprising a binding protein for albumin wherein the albumin binding protein comprises or consists of an amino acid sequence selected from the group of SEQ ID NOs: 1-32, or an amino acid sequence with at least 95% amino acid sequence identity thereto, wherein the K_{D} to HSA is below 25 nM. The present invention provides a fusion protein comprising a binding protein for albumin wherein the albumin binding protein comprises or consists of an amino acid sequence with at least 95% amino acid sequence identity to anyone selected from the group of SEQ ID NOs: 1-32, wherein the K_{D} to HSA is below 25 nM. In preferred embodiments, the 95% sequence identity allows for up to two modifications, in particular substitutions, in the amino acid sequence of any one of SEQ ID NOs: 1-32 at positions other than positions 9, 15, 17, 19, 20, 22, 23, 26, 27, 31, 32, 35, 39, 40, 42, and 44. In various preferred embodiments, the 95% sequence identity allows for up to two modifications, in particular substitutions, in the amino acid sequence of any one of SEQ ID NOs: 1-32 at any of positions 20, 21, and/or 33.

Embodiments of the invention relate to a fusion protein comprising a binding protein for albumin wherein the albumin binding protein consists of an amino acid sequence selected from the group of SEQ ID NOs: 1-32, or an amino acid sequence with 1 or 2 substitutions, deletions, or insertions thereto wherein the amino acids of positions 1-14 consist of amino acids LAEAKVLAIKELDK (SEQ ID NO: 52) or of amino acids LAEAKVLALKELDK (SEQ ID NO: 51).

Some embodiments of the invention relate to a fusion protein comprising a binding protein for albumin wherein the albumin binding protein consists of an amino acid sequence selected from the group of SEQ ID NOs: 1-32, or an amino acid sequence with 1 or 2 substitutions, deletions, or insertions thereto but wherein the amino acids of positions 1-14 are LAEAKVLAIKELDK. Some embodiments of the invention relate to a fusion protein comprising a binding protein for albumin wherein the albumin binding protein consists of an amino acid sequence with at least 95%, at least 97% or 100% identity to SEQ ID NOs: 1-32 wherein the amino acids of positions 1-14 are LAEAKVLALKELDK.

Some embodiments of the invention relate to a fusion protein comprising a binding protein for albumin wherein the albumin binding protein is binding to mammalian serum albumin. Some embodiments of the invention relate to a fusion protein comprising a binding protein for albumin wherein the albumin binding protein is binding to human serum albumin (HSA) and/or mouse serum albumin (MSA).

In one embodiment of the invention, the fusion protein comprises an albumin-binding domain as described above and a tumor target-binding domain.

Some embodiments of the invention relate to a fusion protein comprising a binding protein for albumin and additionally at least one targeting moiety selected from a non-immunoglobulin protein or antibody or antibody fragment. In some embodiments, the targeting moiety is capable of binding to targeting protein with a dissociation constant K_{D} of 100 nM or less. Some embodiments of the invention relate to a fusion protein comprising a binding protein for albumin and additionally at least one targeting moiety selected from a non-immunoglobulin protein or antibody or antibody fragment. In some embodiments, the targeting moiety is capable of binding to targeting protein with a dissociation constant K_{D} of 10 nM or less.

Some embodiments of the invention relate to a fusion protein comprising a binding protein for albumin and additionally at least one targeting moiety selected from a non-immunoglobulin protein, wherein the non-immunoglobulin protein a ubiquitin mutein, a mutein of domains of protein A, an ankyrin repeat protein mutein, lipocalin mutein, a mutein of human Fyn SH3 domain, a mutein of the tenth domain of human fibronectin, a mutein of FN3 domain, a mutein of Kunitz domains, a Sac7d mutein, a chagasin mutein, a mutein of multimerized low density lipoprotein receptor-A, a mutein of cysteine-knot miniprotein, a mutein of Stefin, a mutein of Armadillo-repeat protein, a mutein of tetranectin, a mutein of C-type lectin domain, or a mutein of CTLA4, or antibodies or antibody fragments or sa ingle domain antibody or single chain variable fragments of antibodies.

Some embodiments of the invention relate to a fusion protein comprising a binding protein for albumin and additionally at least one targeting moiety selected from a non-immunoglobulin protein, wherein the non-immunoglobulin protein is a ubiquitin mutein exhibiting 80% to 94% identity to ubiquitin, preferably at least 85% to 94% identity to ubiquitin (SEQ ID NO: 43). In various embodiments, the tumor targeting moiety is a ubiquitin mutein exhibiting 80% to 94% identity to ubiquitin, preferably at least 85% to 94% identity to ubiquitin, and having a binding affinity of less than 100 nM for a protein expressed by a tumor as described elsewhere herein. In various embodiments, the tumor targeting moiety is a ubiquitin mutein exhibiting 80% to 94% identity to ubiquitin, preferably at least 85% to 94% identity to ubiquitin, and having a binding affinity of at least 25 nM, or at least 100 nM, or at least 200 nM, or at least 500 nM, for a protein expressed by a tumor as described elsewhere herein. As will be appreciated by a person of ordinary skill in the art, the terms “less than” and “at least” may be used interchangeably herein since they both refer to the binding affinity of the tumor targeting moiety for a protein expressed by a tumor. The same applies with regard to the terms “less than” and “at least” in the context of the binding affinity of the serum albumin binding protein/domain to serum albumin as described elsewhere herein.

In various embodiments, the tumor targeting moiety is a ubiquitin mutein exhibiting 80% to 94% identity to ubiquitin, preferably at least 85% to 94% identity to ubiquitin, and having a binding affinity of less than 100 nM for any one of PD-L1, PSMA, FOLR1, FOLR2, EGFR, Her2, ED-B, or FAP.

In various embodiments, the tumor targeting moiety is a ubiquitin mutein exhibiting 80% to 94% identity to ubiquitin, preferably at least 85% to 94% identity to ubiquitin, and having a binding affinity of at least 25 nM, or at least 100 nM, or at least 200 nM, or at least 500 nM, for any one of PD-L1, PSMA, FOLR1, FOLR2, EGFR, Her2, ED-B, or FAP.

Some embodiments of the invention relate to a fusion protein comprising a binding protein for albumin and additionally at least one targeting moiety, wherein the targeting moiety binds to a protein tumor-specific target with a dissociation constant K_{D} of 100 nM or less, preferably 10 nM or less. In some embodiments, the targeting moiety binds to a protein that is expressed in tumors or on tumor cells. In specific embodiments, the tumor specific targeting moiety is specific for the extracellular domain of Her2. In specific embodiments, the tumor specific targeting moiety is specific for the extracellular domain of FAP. In specific embodiments, the tumor specific targeting moiety is specific for ED-B.

The further characterization of the fusion protein of the invention can be performed in the form of the isolated, soluble proteins. The appropriate methods are known to those skilled in the art or described in the literature. Such methods include the determination of physical, biophysical and functional characteristics of the proteins. The affinity and specificity of the fusion proteins can be detected by means of biochemical standard methods as discussed above and in the Examples and as known to those skilled in the art.

In some embodiments, the fusion protein as described herein has a binding affinity (K_{D}) of less than 25 nM for human serum albumin. The fusion proteins bind HSA with measurable binding affinity of less than 25 nM, less than 10 nM, and less than 5 nM, and less than 1 nM. The appropriate methods are known to those skilled in the art or described in the literature. The methods for determining the binding affinities are known per se and can be selected for instance from the following methods known in the art: enzyme-linked immunosorbent assay (ELISA), surface plasmon resonance (SPR), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Some of the methods are described in the Examples below. Typically, the dissociation constant K_{D} is determined at the range of temperatures between 20° C. and 30° C., for example at 20° C., 25° C., or 30° C. Typically, the dissociation constant K_{D} is determined at a pH of 7.3. The lower the K_{D} value, the greater the binding affinity of the biomolecule for its binding partner. The higher the K_{D} value, the weaker the binding partners bind to each other. A binding to HSA with K_{D} less than 25 nM, preferably less than 10 nM, may be important for targeted therapeutic applications for cancer treatment. Importantly, a fusion protein with HSA binding with a K_{D} less than 25 nM, preferably less than 10 nM, has an extended half-life as compared to the targeting moiety without fusion to the serum albumin binding domain.

In various embodiments of the present invention, the binding affinity of the serum albumin binding protein to serum albumin is less than 25 nM, preferably less than 10 nM, or less than 5 nM, or less than 1 nM, at pH 7.3. In various other embodiments of the present invention, the binding affinity of the serum albumin binding protein to serum albumin is less than 25 nM, preferably less than 10 nM, or less than 5 nM, or less than 1 nM, as determined by SPR, preferably less than 25 nM, or less than 10 nM, or less than 5 nM, or less than 1 nM, as determined by SPR at pH 7.3. In various embodiments of the present invention, the binding affinity of the tumor-targeting moiety to a protein expressed by a tumor is less than 100 nM, or less than 50 nM, or less than 20 nM, at pH 7.3. In various embodiments of the present invention, the binding affinity of the tumor-targeting moiety to a protein expressed by a tumor is less than 100 nM, or less than 50 nM, or less than 20 nM, as determined by SPR, preferably less than 100 nM, or less than 50 nM, or less than 20 nM, as determined by SPR at pH 7.3.

In various embodiments, the fusion protein comprising the albumin binding domain and a tumor-specific binding protein with high affinity for serum albumin has a half-life in (human or mouse) serum or blood of at least or more than about 17 hours, preferably at least or more than about 24 hours. In preferred embodiments, the tumor-specific binding protein is a non-immunoglobulin protein, more preferably the tumor-specific binding protein is a non-immunoglobulin protein comprising a ubiquitin mutein exhibiting 80% to 94% identity to ubiquitin (SEQ ID NO: 43), even more preferably the tumor-specific binding protein is a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, and/or ED-B.

In various embodiments, the fusion protein comprising the albumin binding domain and a tumor-specific binding protein with high affinity for serum albumin has a half-life in (human or mouse) serum or blood of at least or more than about 30 hours, preferably at least or more than about 36 hours. In preferred embodiments, the tumor-specific binding protein is a non-immunoglobulin protein, more preferably the tumor-specific binding protein is a non-immunoglobulin protein comprising a ubiquitin mutein exhibiting 80% to 94% identity to ubiquitin (SEQ ID NO: 43), even more preferably the tumor-specific binding protein is a non-immunoglobulin protein comprising a ubiquitin mutein exhibiting 80% to 94% identity to ubiquitin (SEQ ID NO: 43) and binding to a protein expressed by tumors such PD-L1, PSMA, FOLR1, FOLR2, EGFR, Her2, FAP, and/or ED-B.

In various embodiments, a fusion protein of the invention comprises, or consists of, (no more than) one serum albumin binding domain and (no more than) one tumor targeting moiety (or tumor-specific binding protein). Such fusion proteins of the invention may have a half-life in (human or mouse) serum or blood of at least (or more than) about 17 hours, about 24 hours, about 30 hours, or about 36 hours. In preferred embodiments, the (no more than) one tumor targeting moiety is a non-immunoglobulin protein, more preferably the (no more than) one tumor targeting moiety is a non-immunoglobulin protein comprising a ubiquitin mutein exhibiting 80% to 94% identity to ubiquitin (SEQ ID NO: 43), even more preferably the (no more than) one tumor targeting moiety is a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various embodiments, a fusion protein of the invention comprises, or consist of, (i) (no more than) one serum albumin binding domain with at least 90% identity to the amino acid sequence of SEQ ID NO: 9, and (ii) (no more than) one tumor targeting moiety (or tumor-specific binding protein) with at least 90% identity to the amino acid sequence of SEQ ID NO: 42. Preferably, the (no more than) one serum albumin binding domain has at least 93% or 94% identity, more preferably 95% or 96% identity, still more preferably 97% or 98% identity, or even 100% identity to the amino acid sequence of SEQ ID NO: 9, and/or the (no more than) one tumor targeting moiety exhibits at least 93% or 94% identity, more preferably 95% or 96% identity, still more preferably 97% or 98% identity, or even 100% identity to the amino acid sequence of SEQ ID NO: 42. Such fusion proteins of the invention may have a half-life in (human or mouse) serum or blood of at least (or more than) about 17 hours, at least (or more than) about 24 hours, at least (or more than) about 30 hours, or at least (or more than) about 36 hours. Such fusion proteins of the invention may have an affinity for MSA of less than 100 nM, preferably less than 50 nM, more preferably less than 40 nM (as determined by SPR; pH 7.3). Such fusion proteins of the invention may have an affinity for FAP, of less than 10 nM, preferably ≤6 nM (as determined by SPR). In various embodiments, the at least 90% sequence identity in regard to SEQ ID NO: 9 means substitutions in the amino acid sequence of SEQ ID NO: 9 at positions other than positions 9, 15, 17, 19, 20, 22, 23, 26, 27, 31, 32, 35, 39, 40, 42, and 44. In various preferred embodiments, the at least 90% sequence identity in regard to SEQ ID NO: 9 means substitutions in the amino acid sequence of SEQ ID NO: 9 at positions including any of positions 20, 21, and/or 33. Further, in preferred embodiments, the (no more than) one tumor targeting moiety is a non-immunoglobulin protein, more preferably the (no more than) one tumor targeting moiety is a non-immunoglobulin protein comprising a ubiquitin mutein exhibiting 80% to 94% identity to ubiquitin (SEQ ID NO: 43), even more preferably the (no more than) one tumor targeting moiety is a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various embodiments, a fusion protein of the invention comprises, or consists of, (no more than) one serum albumin binding domain and (at least) two tumor targeting moieties (or tumor-specific binding proteins). Such fusion proteins of the invention may have a half-life in (human or mouse) serum or blood of at least (or more than) about 17 hours. Such fusion proteins of the invention may have a half-life in (human or mouse) serum or blood of at least (or more than) about 24 or 26 hours or at least (or more than) about 30 hours. In various embodiments, such fusion proteins of the invention may have a half-life in (human or mouse) serum or blood of at least (or more than) about 28 or 29 hours. In various other embodiments, such fusion proteins of the invention may have a half-life in (human or mouse) serum or blood of at least (or more than) about 36 hours. In preferred embodiments, the (at least) two tumor targeting moieties are each a non-immunoglobulin protein, more preferably the (at least) two tumor targeting moieties are each a non-immunoglobulin protein comprising a ubiquitin mutein exhibiting 80% to 94% identity to ubiquitin (SEQ ID NO: 43), even more preferably the (at least) two tumor targeting moieties are each a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various embodiments, a fusion protein of the invention comprises, or consist of, (i) (no more than) one serum albumin binding domain with at least 90% identity to the amino acid sequence of SEQ ID NO: 9, and (ii) (at least) two tumor targeting moieties (or tumor-specific binding proteins), wherein each of the (at least) two tumor targeting moieties exhibits at least 90% identity to the amino acid sequence of SEQ ID NO: 42. Preferably, the (no more than) one serum albumin binding domain has at least 93% or 94% identity, more preferably 95% or 96% identity, still more preferably 97% or 98% identity, or even 100% identity to the amino acid sequence of SEQ ID NO: 9, and/or the (at least) two tumor targeting moieties (or tumor-specific binding proteins) each exhibit at least 93% or 94% identity, more preferably 95% or 96% identity, still more preferably 97% or 98% identity, or even 100% identity to the amino acid sequence of SEQ ID NO: 42. Such fusion proteins of the invention may have a half-life in (human or mouse) serum or blood of at least (or more than) about 24 or 26 hours or at least (or more than) about 30 hours. In various other embodiments, such fusion proteins of the invention may have a half-life in (human or mouse) serum or blood of at least (or more than) about 36 hours. In various embodiments, such fusion proteins of the invention may have a half-life in (human or mouse) serum or blood of at least (or more than) about 28 or 29 hours. Such fusion proteins of the invention may have an affinity for MSA of less than 100 nM, preferably less than 70 nM (as determined by SPR; pH 7.3). Such fusion proteins of the invention may have an affinity for FAP, of less than 10 nM, preferably ≤1 nM (as determined by SPR). In various embodiments, the at least 90% sequence identity in regard to SEQ ID NO: 9 means substitutions in the amino acid sequence of SEQ ID NO: 9 at positions other than positions 9, 15, 17, 19, 20, 22, 23, 26, 27, 31, 32, 35, 39, 40, 42, and 44. In various preferred embodiments, the at least 90% sequence identity in regard to SEQ ID NO: 9 means substitutions in the amino acid sequence of SEQ ID NO: 9 at positions including any of positions 20, 21, and/or 33. Further, in preferred embodiments, the (at least) two tumor targeting moieties are each a non-immunoglobulin protein, more preferably the (at least) two tumor targeting moieties are each a non-immunoglobulin protein comprising a ubiquitin mutein exhibiting 80% to 94% identity to ubiquitin (SEQ ID NO: 43), even more preferably the (at least) two tumor targeting moieties are each a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various embodiments, a fusion protein of the invention comprises an amino acid sequence with at least 90% identity to the amino acid sequence of SEQ ID NO: 54 (220519). Preferably, the fusion protein has at least 93% or 94% identity, more preferably 95% or 96% identity, still more preferably 97% or 98% identity, or even 100% identity to the amino acid sequence of SEQ ID NO: 54. Such fusion proteins of the invention may have a half-life in (human or mouse) serum or blood of at least (or more than) about 24 hours or at least (or more than) about 30 hours. In various embodiments, such fusion proteins of the invention may have a half-life in (human or mouse) serum or blood of at least (or more than) about 28 or 29 hours. For example, SEQ ID NO: 54 (220519, comprising albumin binding domain of SEQ ID NO: 9) has a half-life in blood of about 28 hours. For example, FAP specific fusion protein 224216 comprising albumin binding domain of SEQ ID NO: 6 (c20) and FAP specific binding protein (dimer of SEQ ID NO: 42) has a half-life in blood of about 29 hours. In various other embodiments, such fusion proteins of the invention may have a half-life in (human or mouse) serum or blood of at least (or more than) about 36 hours. Such fusion proteins of the invention may have an affinity for MSA of less than 100 nM, preferably less than 70 nM (as determined by SPR; pH 7.3). Such fusion proteins of the invention may have an affinity for FAP, of less than 10 nM, preferably ≤1 nM (as determined by SPR).

In various other embodiments, a fusion protein of the invention comprises, or consist of, (at least) two serum albumin binding domains and (no more than) one tumor targeting moiety (or tumor-specific binding protein). Such fusion proteins of the invention may have a half-life in (human or mouse) serum or blood of at least (or more than) about 41 hours. In preferred embodiments, the (no more than) one tumor targeting moiety (or tumor-specific binding protein) is a non-immunoglobulin protein, more preferably the (no more than) one tumor targeting moiety (or tumor-specific binding protein) is a non-immunoglobulin protein comprising a ubiquitin mutein exhibiting 80% to 94% identity to ubiquitin (SEQ ID NO: 43), even more preferably the (no more than) one tumor targeting moiety (or tumor-specific binding protein) is a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various other embodiments, a fusion protein of the invention comprises, or consists of, (at least) two serum albumin binding domains and (at least) two tumor targeting moieties (or tumor-specific binding proteins). Such fusion proteins of the invention may have a half-life in (human or mouse) serum or blood of at least (or more than) about 36 hours, in various embodiments at least (or more than) about 39 hours. In some embodiments, such fusion proteins of the invention may have a half-life in (human or mouse) serum or blood of at least (or more than) about 47 hours. In preferred embodiments, the (at least) two tumor targeting moieties are each a non-immunoglobulin protein, more preferably the (at least) two tumor targeting moieties are each a non-immunoglobulin protein comprising a ubiquitin mutein exhibiting 80% to 94% identity to ubiquitin (SEQ ID NO: 43), even more preferably the (at least) two tumor targeting moieties are each a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various embodiments, a fusion protein of the invention comprises, or consist of, (i) (at least) two serum albumin binding domains, wherein each of the (at least) two serum albumin binding domains exhibits at least 90% identity to the amino acid sequence of SEQ ID NO: 9 (d01), and (ii) and (at least) two tumor targeting moieties (or tumor-specific binding proteins), wherein each of the (at least) two tumor targeting moieties exhibits at least 90% identity to the amino acid sequence of SEQ ID NO: 42. Preferably, the (at least) two serum albumin binding domains each exhibit at least 93% or 94% identity, more preferably 95% or 96% identity, still more preferably 97% or 98% identity, or even 100% identity to the amino acid sequence of SEQ ID NO: 9, and/or the (at least) two tumor targeting moieties (or tumor-specific binding proteins) each exhibit at least 93% or 94% identity, more preferably 95% or 96% identity, still more preferably 97% or 98% identity, or even 100% identity to the amino acid sequence of SEQ ID NO: 42. Such fusion proteins of the invention may have a half-life in (human or mouse) serum or blood of at least (or more than) about 35 or 36 hours, in various embodiments at least (or more than) about 39 hours. In some embodiments, such fusion proteins of the invention may have a half-life in (human or mouse) serum or blood of at least (or more than) about 47 hours. Such fusion proteins of the invention may have an affinity for MSA of less than 10 nM, preferably ≤1 nM (as determined by SPR; pH 7.3). Such fusion proteins of the invention may have an affinity for FAP, of less than 10 nM, preferably ≤1 nM, more preferably ≤0.5 nM (as determined by SPR). In various embodiments, the at least 90% sequence identity in regard to SEQ ID NO: 9 means substitutions in the amino acid sequence of SEQ ID NO: 9 at positions other than positions 9, 15, 17, 19, 20, 22, 23, 26, 27, 31, 32, 35, 39, 40, 42, and 44. In various preferred embodiments, the at least 90% sequence identity in regard to SEQ ID NO: 9 means substitutions in the amino acid sequence of SEQ ID NO: 9 at positions including any of positions 20, 21, and/or 33. Further, in preferred embodiments, the (at least) two tumor targeting moieties are each a non-immunoglobulin protein, more preferably the (at least) two tumor targeting moieties are each a non-immunoglobulin protein comprising a ubiquitin mutein exhibiting 80% to 94% identity to ubiquitin (SEQ ID NO: 43), even more preferably the (at least) two tumor targeting moieties are each a non-immunoglobulin protein comprising a ubiquitin mutein exhibiting 80% to 94% identity to ubiquitin (SEQ ID NO: 43) and binding to a protein expressed by tumors such as PD-L1, PSMA, FOLR1, FOLR2, EGFR, Her2, FAP, or ED-B.

In various embodiments, a fusion protein of the invention comprises an amino acid sequence with at least 90% identity to the amino acid sequence of SEQ ID NO: 47 (220521). Preferably, the fusion protein has at least 93% or 94% identity, more preferably 95% or 96% identity, still more preferably 97% or 98% identity, or even 100% identity to the amino acid sequence of SEQ ID NO: 47. Such fusion proteins of the invention may have a half-life in (human or mouse) serum or blood of at least (or more than) about 35 or 36 hours, in various embodiments at least (or more than) about 39 hours, more preferably at least (or more than) about 47 hours. Such fusion proteins of the invention may have an affinity for MSA of less than 10 nM, preferably ≤1 nM (as determined by SPR; pH 7.3). Such fusion proteins of the invention may have an affinity for FAP, of less than 10 nM, preferably ≤1 nM, more preferably ≤0.5 nM (as determined by SPR).

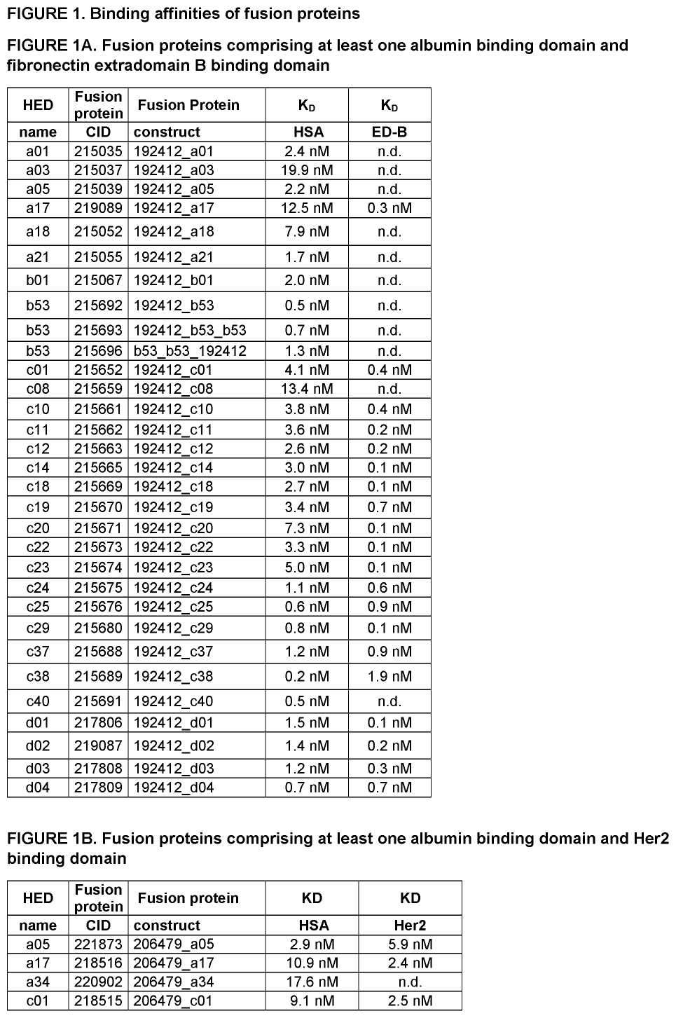

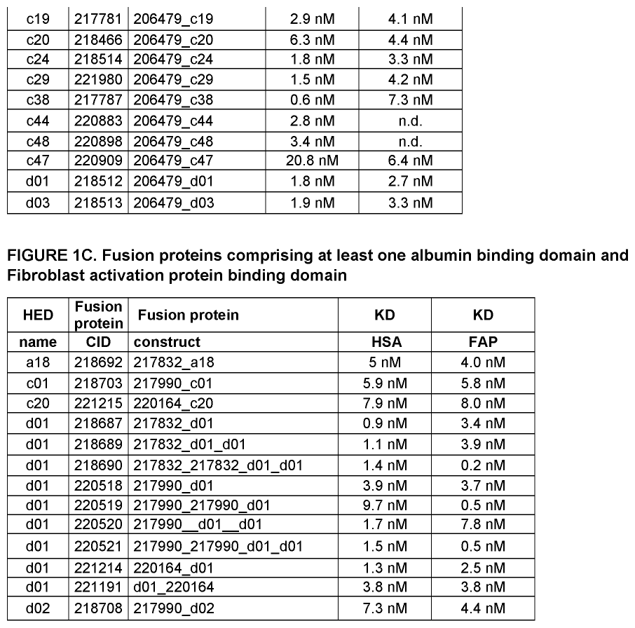

Some embodiments relate to the fusion protein as described above for use in treatment of medical disorders such as cancer. In the treatment of cancer it is of great importance that the therapeutic moiety (targeting moiety) is specifically directed to the tumor cell of interest and wherein the half-life of the targeting moiety is long (e.g. longer than 17 h or 24 h). This can be achieved by a use of the fusion protein of the invention comprising the targeting moiety and the albumin binding domain of the invention having a dissociation constant K_{D} to human HSA of less than 25 nM as determined with surface plasmon resonance (see FIG. 1). In some embodiments, the half-life of a fusion protein as described herein is between 17-40 hours, in various preferred embodiments between 17-39 hours, in various further embodiments between 28-39 hours. In some embodiments, the half-life of a fusion protein as described herein is around 36 hours. In some other embodiments, the half-life of a fusion protein as described herein is around 39 hours. In some further embodiments, the half-life of a fusion protein as described herein is around 17 hours. Still further, in some embodiments, the half-life of a fusion protein as described herein is around 28 or 29 hours. For diagnostic and therapeutic applications, it might be of great importance to have molecules with extended half-life. Some embodiments relate to a fusion protein comprising an albumin binding domain of SEQ ID NOs: 33, or at least 95% or at least 97% identical proteins thereto. Some embodiments relate to a fusion protein comprising an albumin binding domain selected from SEQ ID NOs: 1-32, or at least 95% or at least 97% identical proteins thereto, provided that the affinity to human serum albumin is in the range of 0.1 nM and 25 nM.

Several techniques for producing fusion proteins with extended half-life are known in the art, for example, direct fusions of the moiety modulating pharmacokinetics with the fusion protein as described above or chemical coupling methods. In some embodiments, the moiety modulating pharmacokinetics can be attached for example directly or at one or several sites of the fusion protein through a peptide linker sequence or through a coupling site as described above.

Fusion Protein that Bind to HSA with Moderate Affinity.

In one embodiment of the invention, the fusion protein comprising the albumin binding domain of the invention and a targeting domain has a dissociation constant K_{D} to HSA of between 25 nM and 3 μM as determined with surface plasmon resonance (see EXAMPLES). A moderate affinity binding of the albumin binding domain to HSA with a K_{D} between 25 nM and 3 μM, or between 50 nM and 3 μM, or between 100 nM and 3 μM, may be important for targeted diagnostic applications for cancer wherein a defined shorter half-life of a fusion protein of the invention is desirable. For example, fusion proteins comprising albumin binding domains c42, c45, c49, or c50 have a half-life of about 6-15 hours. The binding affinity of the albumin binding domain to serum albumin may be in a range between 25 nM and 100 nM, between 25 nM and 200 nM, or between 25 nM and 500 nM.

In various embodiments of the present invention, the binding affinity of the tumor-targeting moiety to a protein expressed by a tumor is at least 25 nM, or at least 100 nM, or at least 200 nM, or at least 500 nM, at pH 7.3. In various embodiments of the present invention, the binding affinity of the tumor-targeting moiety to a protein expressed by a tumor at least 100 nM, or at least 200 nM, or at least 500 nM, as determined by SPR, preferably at least 25 nM, or at least 100 nM, or at least 200 nM, or at least 500 nM, as determined by SPR at pH 7.3. The binding affinity of the tumor-targeting moiety may be in a range between 25 nM and 100 nM, between 25 nM and 200 nM, or between 25 nM and 500 nM.

Some embodiments relate to fusion proteins comprising any one of SEQ ID NOs: 34-38, or at least 95%, 97%, or 100% identical proteins to any one of SEQ ID NOs: 34-38, provided that the affinity to human serum albumin is in the range of 25 nM and 3 μM, or between 50 nM and 3 μM, or between 100 nM and 3 μM, or between 25 nM and 100 nM, or between 25 nM and 200 nM, or between 25 nM and 500 nM.

Accordingly, the present invention provides a fusion protein comprising (a) a binding protein for serum albumin comprising the amino acid sequence of any one of SEQ ID NOs: 34-38, or comprising an amino acid sequence with at least 95% or 97% identity to any one of SEQ ID NOs: 34-38, and having a binding affinity to serum albumin in the range of 25 nM and 3 UM, and (b) a targeting moiety having a binding affinity of at least 25 nM to a protein expressed by tumors. The binding affinity to serum albumin may follow definitions/ranges as described elsewhere herein for serum albumin binding proteins with moderate affinity to serum albumin. For example, the binding affinity may be in a range between 25 nM and 100 nM, between 25 nM and 200 nM, or between 25 nM and 500 nM, or may be between 50 nM and 3 μM, or between 100 nM and 3 μM. The same applies with regard to the binding affinity of the

The present invention provides a fusion protein comprising (a) a binding protein for serum albumin comprising the amino acid sequence of any one of SEQ ID NOs: 34-38, or comprising an amino acid sequence with 1 or 2 substitutions, deletions, or insertions in the amino acid sequence of any one of SEQ ID NOs: 34-38, and having a binding affinity to serum albumin in the range of 25 nM and 3 μM, and (b) a targeting moiety having a binding affinity of at least 25 nM to a protein expressed by tumors. The binding affinity to serum albumin may follow definitions/ranges as described elsewhere herein for serum albumin binding proteins with moderate affinity to serum albumin. For example, the binding affinity may be in a range between 25 nM and 100 nM, between 25 nM and 200 nM, or between 25 nM and 500 nM, or between 50 nM and 3 UM, or between 100 nM and 3 μM.

In various embodiments, the binding affinity of the serum albumin binding protein to serum albumin means a binding affinity as determined at pH 7.3. In various other embodiments of the present invention, the binding affinity of the serum albumin binding protein to serum albumin means a binding affinity as determined by SPR, preferably as determined by SPR at pH 7.3.

In various embodiments, the binding affinity of the tumor-targeting moiety to a protein expressed by a tumor means a binding affinity as determined at pH 7.3. In various embodiments, the binding affinity of the tumor-targeting moiety to a protein expressed by a tumor means a binding affinity as determined by SPR, as determined by SPR at pH 7.3.

In some embodiments, the fusion protein comprises the albumin binding domain wherein the amino acid sequence of the albumin binding domain with 0, 1 or 2 substitutions, deletions, or insertions of SEQ ID NO: 33 and has a Y, G, L, or D in position 20.

In preferred embodiments, the fusion protein comprises the albumin binding domain wherein the amino acid sequence of the albumin binding domain with 0, 1 or 2 substitutions, deletions, or insertions of SEQ ID NO: 6 (c20) and has a G, L, or D in position 20. Specific examples are provided for albumin binding domain of SEQ ID NO: 35 (c45) and SEQ ID NO: 36 (c46) wherein the K_{D} to human HSA is between 25 nM and 3 μM.

In preferred embodiments, the fusion protein comprises the albumin binding domain wherein the amino acid sequence of the albumin binding domain with 0, 1 or 2 substitutions, deletions, or insertions of SEQ ID NO: 7 (c24) and has a G, L, or D in position 20. One example is provided in SEQ ID NO: 37 (c49) wherein the K_{D} to human HSA is between 25 nM and 3 μM.

In preferred embodiments, the fusion protein comprises the albumin binding domain wherein the amino acid sequence of the albumin binding domain with 0, 1 or 2 substitutions, deletions, or insertions of SEQ ID NO: 33 and has a G in position 33 wherein the K_{D} to human HSA is between 25 nM and 3 μM.

In preferred embodiments, the fusion protein comprises the albumin binding domain wherein the amino acid sequence of the albumin binding domain with 0, 1 or 2 substitutions, deletions, or insertions of SEQ ID NO: 33 and has a G in position 33. One example is provided in SEQ ID NO: 38 (c50) wherein the K_{D} to human HSA is between 25 nM and 3 μM.

In preferred embodiments, the fusion protein comprises the albumin binding domain wherein the amino acid sequence of the albumin binding domain with 0, 1 or 2 substitutions, deletions, or insertions of SEQ ID NO: 33 and has a S in position 33. One example is provided in SEQ ID NO: 34 (c42) wherein the K_{D} to human HSA is between 25 nM and 3 μM, or 50 nM and 3 μM, or 100 nM and 3 μM.

In further preferred embodiments, the serum albumin binding protein comprises at positions 1-14 amino acids LAEAKVLALKELDK (SEQ ID NO: 51), or comprises at positions 1-14 amino acids LAEAKVLAIKELDK (SEQ ID NO: 52).

In some embodiments, the fusion protein comprises the albumin binding domain and a tumor-specific binding protein and has a defined serum half-life of 4-15 hours.

In various embodiments, the fusion protein comprising the albumin binding domain and a tumor-specific binding protein with moderate/low affinity for serum albumin (25 nM to 3 μM) has a half-life in (human or mouse) serum or blood of about 15 hours.

Some embodiments relate to the fusion protein as described above for use in diagnosis of medical disorders such as tumors. In the diagnosis of tumors it is of great importance that the therapeutic moiety (targeting moiety) is specifically directed to the cell of interest and wherein the half-life of the targeting moiety is extended by a defined period of time, for example 4 to 15 h. In some embodiments, the half-life is extended by 6 to 12 h. This can be achieved by use of the fusion protein of the invention comprising the targeting moiety and the albumin binding domain of SEQ ID NOs: 34-38 or at least 95% identical thereto having a dissociation constant K_{D} to human HSA of more than 25 nM, preferably more than 50 nM, more preferably more than 100 nM as determined with surface plasmon resonance. The half-life of such fusion protein as described herein is between 4 to 12 hours, in some embodiments 6 to 11 hours.

For diagnostic and therapeutic applications, it might be of great importance to have molecules with defined extended half-life below 12 h. Some embodiments relate to a fusion protein comprising an albumin binding domain selected from SEQ ID NOs: 34-38, or at least 95% or at least 97% identical proteins thereto, more preferably selected from SEQ ID NO: 34-38.

Further moieties. In some embodiments, conjugation of proteinaceous or non-proteinaceous moieties to the fusion protein may be performed applying chemical methods well-known in the art. In some embodiments, coupling chemistry specific for derivatization of cysteine or lysine residues may be applicable. Chemical coupling can be performed by chemistry well known to someone skilled in the art, including but not limited to, substitution, addition or cycloaddition or redox reactions (e.g. disulfide formation).

Some embodiments relate to a fusion protein wherein the fusion protein comprises additionally at least one diagnostically active moiety. Such diagnostically active moiety might be selected from a radionuclide, fluorescent protein, photosensitizer, chelator, dye, or enzyme, or any combination of the above. In other embodiments, the fusion protein further comprises more than one diagnostic moiety. In some embodiments, a fusion protein that comprises at least one diagnostic moiety can be employed, for example, as imaging agent, for example to evaluate presence of tumor cells or metastases, tumor distribution, and/or recurrence of tumor. Methods for detection or monitoring of cancer cells involve imaging methods. Such methods involve imaging cancer cells by, for example, radio-imaging or photoluminescense or fluorescence. Some embodiments refer to methods of imaging at least a portion of a subject comprising administering a composition to the subject, wherein the composition comprises a fusion protein as described herein and an imaging agent. In some embodiments the method is further comprising the step of diagnosing the subject with a target expressing cancer. In some embodiments the method is further comprising the step of monitoring the distribution of the composition within the subject.

Suitable radionuclides for applications in imaging in vivo or in vitro or for radiotherapy include for example but are not limited to the group of gamma-emitting isotopes, the group of positron emitters, the group of beta-emitters, and the group of alpha-emitters. In some embodiments, suitable conjugation partners include chelators such as 1,4,7,10-tetraazacyclododecane-1,4,7,10-tetraacetic acid (DOTA) or diethylene triamine pentaacetic acid (DTPA) or DATA (6-pentanoic acid-6-amino-1,4-diazapine-triacetate or their activated derivatives. In various embodiments, DOTA may be suitable as complexing agent for radioisotopes and other agents for imaging, as described in the Examples in further detail. Some embodiments refer to methods of imaging at least a portion of a subject comprising administering a composition to the subject, wherein the composition comprises a fusion protein as described herein and a radionuclide loaded to a suitable conjugation partner, for example, DOTA. In some embodiments the method is further comprising the step of diagnosing the subject with a target expressing cancer. In some embodiments the method is further comprising the step of monitoring the distribution of the composition within the subject.

In some embodiments, additional amino acids can extend either at the N-terminal end of the fusion protein or the C-terminal end or both. Additional sequences may include for example sequences introduced e.g. for purification or detection. In one embodiment, additional amino acid sequences include one or more peptide sequences that confer an affinity to certain chromatography column materials. Typical examples for such sequences include, without being limiting, Strep-tags, oligohistidine-tags, glutathione S-transferase, maltose-binding protein, inteins, intein fragments, or the albumin-binding domain of protein G.

Use in medicine. Various embodiments relate to the fusion protein as disclosed herein for use in medicine. In one embodiment, the fusion protein is used in medicine to diagnose or treat tumors associated with target (e.g. Her2, FAP, or ED-B) expression. The fusion proteins as disclosed herein allow selective diagnosis and treatment of tumor cells or tumor tissues.

The present invention encompasses a method of diagnosing cancer in a subject, the method comprising obtaining a sample from the subject, and detecting a protein expressed by a tumor cell using the fusion protein of the present invention. The sample may be an invasive or a non-invasive sample. The said detecting is effected ex vivo. Preferably, the subject is a mammalian subject, more preferably the subject is a human subject.

The present invention encompasses a method of treating cancer in a subject, the method comprising administering to a subject in need thereof a fusion protein of the present invention. Preferably, the subject is a mammalian subject, more preferably the subject is a human subject. For example, the membrane protein Her2 is known to be upregulated in tumor cells, resulting in uncontrolled growth of tumor cells and in the formation of metastases. Overexpression of Her2 has been described in a wide variety of cancers. For example, overexpression of Her2 occurs in approximately 15% to 30% of breast cancers and 10% to 30% of gastric/gastroesophageal cancers and has also been observed in other cancers like ovary, endometrium, bladder, lung colon, head and neck. Thus, the pharmaceutical composition comprising the fusion protein as described herein, can be used for treatment of cancer in which Her2 is relevant for the development of the disease including but not limited to particularly breast, ovarian, gastric, but also in lung, head and neck, cervical, prostate, pancreas, and others.

The FAP binding protein as described herein or a fusion protein comprising the FAP binding protein as disclosed herein allow selective diagnosis and treatment of FAP related cancer cells or cancer tissues, for example, from breast, colorectal, pancreatic, lung, brain, intrahepatic bile duct, and ovarian cancers, or tumors that are derived from non-epithelial tissues, such as melanoma and myeloma. FAP binding proteins are used in diagnosis (imaging) and treatment) for most epithelial cancers, including of breast, lung, colorectal and pancreatic carcinomas. FAP is known to be upregulated in tumor cells, possibly resulting in uncontrolled growth of tumor cells and in the formation of metastases. In one embodiment, the FAP binding protein is used to diagnose FAP related tumors by applying in vitro methods.

ED-B specific fusion proteins with coupled chelators, drugs, toxins, and small molecules can be particularly useful for use in the diagnosis or treatment of cancer including breast, ovarian, prostate, non-small cell lung, colorectal, pancreatic, human skin, hepatocellular, intracraneal meningeoma, glioblastoma or for use in the diagnosis or treatment of cardiovascular diseases including atherosclerotic plaques, myocardial infarction or inflammation and others. For example, fusion protein with dyes coupled to the coupling site can be useful in the diagnosis of cancer. For example, fusion protein with chelators coupled to the coupling site can be useful in diagnostic or therapeutic applications; for example, further substances such as radioisotopes can be coupled to chelators, preferably for use in molecular imaging.

One embodiment is a method of diagnosing (including monitoring) a subject having tumors with expression of a specific target, the method of diagnosis (monitoring) comprising administering to the subject the fusion protein as described, optionally conjugated to radioactive isotope. In various embodiments, the fusion protein as disclosed herein may be used for diagnosis of tumors with specific target proteins, optionally wherein the fusion protein is conjugated to a radioactive isotope. In some embodiments, imaging methods using the fusion protein with labels such as radioactive or fluorescent can be employed to visualize the target protein on specific tissues or cells, for example, to evaluate presence of target specific tumor cells, target specific related tumor distribution, recurrence of target specific tumor, and/or to evaluate the response of a patient to a therapeutic treatment.

One embodiment is a method of treating a subject having target specific cancer, the method of treatment comprising administering to the subject the target specific binding protein as described, optionally conjugated to a radioactive isotope and/or a cytotoxic agent. In various embodiments, the fusion protein as disclosed herein may be used for treatment of target specific cancer, optionally wherein the fusion protein is conjugated to a cytotoxic agent and/or to a radioactive isotope. Some embodiments relate to the use of the fusion protein labelled with a suitable radioisotope or cytotoxic compound for treatment of tumor cells, in particular to control or kill target specific tumor cells, for example malignant cells. In one embodiment, curative doses of radiation are selectively delivered to target specific tumor cells but not to normal cells.

Compositions. Various embodiments relate to a composition comprising the fusion protein as disclosed herein. Some embodiments relate to a composition comprising the fusion protein as defined above for use in medicine, preferably for use in the diagnosis or treatment of various tumors as described above. Compositions comprising the fusion protein as described above may be used for clinical applications for both diagnostic and therapeutic purposes. In particular, compositions comprising the fusion protein as described herein may be used for clinical applications for imaging, monitoring, and eliminating or inactivating pathological cells that express the tumor target or neighbored tumor related structures via a bystander effect for the latter. Various embodiments relate to a diagnostic composition for the diagnosis of target specific cancer comprising the fusion protein as defined herein and a diagnostically acceptable carrier and/or diluent. These include for example but are not limited to stabilizing agents, surface-active agents, salts, buffers, coloring agents etc. The compositions can be in the form of a liquid preparation, a lyophilisate, granules, in the form of an emulsion or a liposomal preparation.

The diagnostic composition comprising the fusion protein as described herein can be used for diagnosis of target specific cancer, as described above.

Various embodiments relate to a pharmaceutical (e.g. therapeutical) composition for the treatment of diseases comprising the fusion protein as disclosed herein, and a pharmaceutically (e.g. therapeutically) acceptable carrier and/or diluent. The pharmaceutical (e.g. therapeutical) composition optionally may contain further auxiliary agents and excipients known per se. These include for example but are not limited to stabilizing agents, e.g. radical quencher, surface-active agents, salts, buffers, coloring agents etc.

The pharmaceutical composition comprising the fusion protein as defined herein can be used for treatment of diseases, as described above.

The compositions contain an effective dose of the fusion protein as defined herein. The amount of protein to be administered depends on the organism, the type of disease, the age and weight of the patient and further factors known per se. Depending on the galenic preparation these compositions can be administered parentally by injection or infusion, systemically, intraperitoneally, intramuscularly, subcutaneously, transdermally, or by other conventionally employed methods of application.

The composition can be in the form of a liquid preparation, a lyophilisate, a cream, a lotion for topical application, an aerosol, in the form of powders, granules, in the form of an emulsion or a liposomal preparation. The type of preparation depends on the type of disease, the route of administration, the severity of the disease, the patient and other factors known to those skilled in the art of medicine.

The various components of the composition may be packaged as a kit with instructions for use. Preparation of fusion proteins. fusion proteins as described herein may be prepared by any of the many conventional and well-known techniques such as plain organic synthetic strategies, solid phase-assisted synthesis techniques, fragment ligation techniques or by commercially available automated synthesizers. On the other hand, they may also be prepared by conventional recombinant techniques alone or in combination with conventional synthetic techniques. Furthermore, they may also be prepared by cell-free in vitro transcription/translation.

Some embodiments relate to a nucleic acid molecule encoding the fusion protein as described above.

One embodiment further provides an expression vector comprising said nucleic acid molecule, and a host cell comprising said isolated polynucleotide or the expression vector.

In one embodiment, the present invention is directed to a vector comprising the nucleic acid molecule as described above. A vector means any molecule or entity (e.g., nucleic acid, plasmid, bacteriophage or virus) that can be used to transfer protein coding information into a host cell. In one embodiment, the vector is an expression vector.

In one embodiment, the present invention is related to a host cell or a non-human host comprising the fusion protein as described herein, a nucleic acid as described herein, and/or a vector as described herein.

Various embodiments relate to a method for the production of a fusion protein as disclosed herein comprising culturing of a host cell under suitable conditions which allow expression of said fusion protein and optionally isolating said fusion protein.

For example, one or more polynucleotides which encode for the fusion protein may be expressed in a suitable host and the produced fusion protein can be isolated. A host cell comprises said nucleic acid molecule or vector. Suitable host cells include prokaryotes or eukaryotes. A vector means any molecule or entity (e.g., nucleic acid, plasmid, bacteriophage or virus) that can be used to transfer protein coding information into a host cell. Various cell culture systems, for example but not limited to mammalian, yeast, plant, or insect, can also be employed to express recombinant proteins. Suitable conditions for culturing prokaryotic or eukaryotic host cells are well known to the person skilled in the art. Cultivation of cells and protein expression for the purpose of protein production can be performed at any scale, starting from small volume shaker flasks to large fermenters, applying technologies well-known to any skilled in the art.

One embodiment is directed to a method for the preparation of a binding protein as detailed above, said method comprising the following steps: (a) preparing a nucleic acid encoding a fusion protein as defined herein; (b) introducing said nucleic acid into an expression vector; (c) introducing said expression vector into a host cell; (d) cultivating the host cell; (e) subjecting the host cell to culturing conditions under which a fusion protein is expressed, thereby producing a fusion protein as defined herein; (f) optionally isolating the fusion protein produced in step (e); and (g) optionally conjugating the fusion protein with further functional moieties as defined herein. In general, isolation of purified fusion protein from the cultivation mixture can be performed applying conventional methods and technologies well known in the art, such as centrifugation, precipitation, flocculation, different embodiments of chromatography, filtration, dialysis, concentration and combinations thereof, and others. Chromatographic methods are well-known in the art and comprise without limitation ion exchange chromatography, gel filtration chromatography (size exclusion chromatography), hydrophobic interaction chromatography, or affinity chromatography.

For simplified purification, the fusion protein can be fused to other peptide sequences having an increased affinity to separation materials. Preferably, such fusions are selected that do not have a detrimental effect on the functionality of the fusion protein or can be separated after the purification due to the introduction of specific protease cleavage sites. Such methods are also known to those skilled in the art.

Further aspects of the present invention include:

1. A binding protein for serum albumin comprising the amino acid sequence of SEQ ID NO: 33, or an amino acid sequence with 1 or 2 substitutions, deletions, or insertions in the amino acid sequence of SEQ ID NO: 33, and having a binding affinity to serum albumin of less than 25 nM.

2. A binding protein for serum albumin (or a serum albumin binding protein according to item 1), wherein the serum albumin binding protein comprises an amino acid sequence selected from the group of SEQ ID NOs: 1-32, or comprises an amino acid sequence with 1 or 2 substitutions, deletions, or insertions in the amino acid sequence of SEQ ID NOs: 1-32, and has a binding affinity to serum albumin of less than 25 nM.

3. The serum albumin binding protein according to item 1 or 2, comprising a binding protein for human serum albumin or mouse serum albumin.

4. The serum albumin binding protein according to any one of items 1-3, wherein the 1 or 2 substitutions, deletions, or insertions to any of SEQ ID NOs: 1-33 are made at positions other than positions 9, 15, 17, 19, 20, 22, 23, 26, 27, 31, 32, 35, 39, 40, 42, and 44. Accordingly, in such embodiments, the substitutions in positions 9, 15, 17, 19, 20, 22, 23, 26, 27, 31, 32, 35, 39, 40, 42, and 44 as defined in SEQ ID NO: 33 remain unaffected. In other preferred embodiments, the 1 or 2 substitutions, deletions or insertions in the amino acid sequence of SEQ ID NO: 33 are made at any one of positions 20, 21 and/or 33 of SEQ ID NO: 33, as described herein above. Accordingly, in such embodiments, the substitutions in positions 9, 15, 17, 19, 22, 23, 26, 27, 31, 32, 35, 39, 40, 42, and 44 according to SEQ ID NO: 33 remain unaffected.

In various preferred embodiments, the 1 or 2 substitutions, deletions, or insertions in the amino acid sequence of SEQ ID NO: 33 are 1 or 2 substitutions or deletions in the amino acid sequence of SEQ ID NO: 33, more preferably 1 or 2 substitutions in the amino acid sequence of SEQ ID NO: 33.

In preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₃₁ is I, X₃₂ is E, X₃₉ is D, and X₄₀ is E (as shown for SEQ ID NO: 6).

In preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₃₁ is V, X₃₂ is E, X₃₉ is E, X₄₀ is A (as shown for SEQ ID NO: 9).

In further preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₉ is L, and more preferably the amino acids of positions 1-14 consist of amino acids LAEAKVLALKELDK (SEQ ID NO: 51). In other further preferred embodiments, the serum albumin binding protein comprises the amino acid sequence of SEQ ID NO: 33, or an amino acid sequence with 1 or 2 substitutions, deletions, or insertions in the amino acid sequence of SEQ ID NO: 33, wherein X₉ is I, and more preferably the amino acids of positions 1-14 consist of amino acids LAEAKVLAIKELDK (SEQ ID NO: 52).

5. The serum albumin binding protein according to any one of items 1-3, wherein the amino acid in positions 1, 2, 3, 4, 5, 6, 7, 8, 10, 11, 12, 13, 14, 16, 18, 21, 24, 25, 28, 29, 30, 33, 34, 36, 37, 38, 41, 43 of any one of SEQ ID NOs: 1-33 are not substituted or deleted. In such embodiments, the 1 or 2 substitutions, deletions, or insertions to any of SEQ ID NOs: 1-33 are made at 1 or 2 positions selected from positions 9, 15, 17, 19, 20, 22, 23, 26, 27, 31, 32, 35, 39, 40, 42, and 44 of any of SEQ ID NOs: 1-33, wherein in case of “1 or 2 substitutions”, these differ from the substitutions defined for these positions in SEQ ID NO: 33. In preferred embodiments, the “1 or 2 substitutions” include a substitution at position 20 of SEQ ID NO: 33, wherein such a substitution preferably still provides for an aromatic amino acid, as described elsewhere herein.

6. The serum albumin protein according to item 1, wherein the binding protein for albumin comprises an amino acid sequence with at least 95% sequence identity to the amino acid sequence of any one of SEQ ID NOs: 1-33. The at least 95% sequence identity allows for up to two modifications, in particular substitutions, in the amino acid sequence of SEQ ID NO: 33 at positions other than positions 9, 15, 17, 19, 20, 22, 23, 26, 27, 31, 32, 35, 39, 40, 42, and 44. Accordingly, in such embodiments, the substitutions in positions 9, 15, 17, 19, 20, 22, 23, 26, 27, 31, 32, 35, 39, 40, 42, and 44 according to SEQ ID NO: 33 remain unaffected. In various preferred embodiments, the binding protein for albumin comprises an amino acid sequence with at least 95% sequence identity to the amino acid sequence of SEQ ID NO: 33, wherein the 95% sequence identity allows for up to two modifications, in particular substitutions, in the amino acid sequence of SEQ ID NO: 33 at any of positions 20, 21, and/or 33 of SEQ ID NO: 33. Accordingly, in such embodiments, the substitutions in positions 9, 15, 17, 19, 22, 23, 26, 27, 31, 32, 35, 39, 40, 42, and 44 according to SEQ ID NO: 33 remain unaffected.

In preferred embodiments, the at least 95% sequence identity allows for up to two modifications, in particular substitutions, in the amino acid sequence of any one of SEQ ID NOs: 1-32 at positions other than positions 9, 15, 17, 19, 20, 22, 23, 26, 27, 31, 32, 35, 39, 40, 42, and 44. In various preferred embodiments, the at least 95% sequence identity allows for up to two modifications, in particular substitutions, in the amino acid sequence of any one of SEQ ID NOs: 1-32 at any of positions 20, 21, and/or 33.

7. The serum albumin binding protein according to any one of items 1-6, for use in diagnostics or therapy.

8. A nucleic acid molecule encoding the serum albumin binding protein according to any one of items 1-6.

9. A vector comprising the nucleic acid molecule of item 8.

10. A host cell or a non-human host comprising the serum albumin binding protein as defined in any one of items 1 to 6, a nucleic acid as defined in item 8, and/or a vector of item 9.

11. A composition comprising the serum albumin binding protein as defined in any one of items 1-6, a nucleic acid as defined in item 8, and/or a vector of item 9.

12. A method for the production of the serum albumin binding protein as defined in any one of items 1-6, comprising culturing of the host cell of item 10 under conditions suitable to obtain said serum albumin binding protein, and optionally isolating said serum albumin binding protein.

13. A binding protein for serum albumin comprising the amino acid sequence of any one of SEQ ID NOs: 34-38, or an amino acid sequence with at least 95% or 97% identity to any one of SEQ ID NOs: 34-38, and having a binding affinity to serum albumin in the range of 25 nM and 3 μM.

14. The binding affinity to serum albumin may follow definitions/ranges as described elsewhere herein for serum albumin binding proteins with moderate affinity to serum albumin. For example, the binding affinity may be in a range between 25 nM and 100 nM, between 25 nM and 200 nM, or between 25 nM and 500 nM, or may be between 50 nM and 3 μM, or between 100 nM and 3 μM.

15. A binding protein for serum albumin (or a serum albumin binding protein according to item 13), wherein the serum albumin binding protein comprises an amino acid sequence selected from the group of any one of SEQ ID NOs: 34-38, or comprising an amino acid sequence with 1 or 2 substitutions, deletions, or insertions in the amino acid sequence of of any one of SEQ ID NOs: 34-38, and having a binding affinity to serum albumin in the range of 25 nM and 3 μM. The binding affinity to serum albumin may follow definitions/ranges as described elsewhere herein for serum albumin binding proteins with moderate affinity to serum albumin. For example, the binding affinity may be in a range between 25 nM and 100 nM, between 25 nM and 200 nM, or between 25 nM and 500 nM, or may be between 50 nM and 3 μM, or between 100 nM and 3 μM.

16. The serum albumin binding protein according to item 13 or 14, comprising a binding protein for human serum albumin or mouse serum albumin.

17. The serum albumin binding protein according to any one of items 13-15, wherein the amino acids of positions 1-14 comprise or consist of amino acids LAEAKVLALKELDK (SEQ ID NO: 51), or comprise or consist of amino acids LAEAKVLAIKELDK (SEQ ID NO: 52).

18. The serum albumin binding protein according to any one of items 13-16, for use in diagnostics or therapy.

19. A nucleic acid molecule encoding the serum albumin binding protein according to any one of items 13-16.

20. A vector comprising the nucleic acid molecule of item 18.

21. A host cell or a non-human host comprising the serum albumin binding protein as defined in any one of items 13-16, a nucleic acid as defined in item 18, and/or a vector of item 19.

22. A composition comprising the serum albumin binding protein as defined in any one of items 13-16, a nucleic acid as defined in item 18, and/or a vector of item 19.

23. A method for the production of the serum albumin binding protein as defined in any one of items 13-16, comprising culturing of the host cell of item 20 under conditions suitable to obtain said serum albumin binding protein, and optionally isolating said serum albumin binding protein.

### EXAMPLES

The following Examples are provided for further illustration of the invention. The invention, however, is not limited thereto, and the following Examples merely show the practicability of the invention on the basis of the above description. For a complete disclosure of the invention reference is made also to the literature cited in the application which is incorporated completely into the application by reference.

### Example 1. Expression and Purification of Fusion Proteins

The genes for fusion proteins were cloned into an expression vector using standard methods, purified and analyzed as described below. All fusion proteins containing a Strep tag were expressed in Escherichia coli and highly purified by affinity chromatography and gel filtration. After affinity chromatography using a Strep-Tactin® Superflow® high capacity column the eluted proteins were applied to a size exclusion chromatography (Superdex™ 75 HiLoad 16/600, Superdex™ 200 HiLoad 16/600 or Sephacryl S200HR 16/600 column) using an ÄKTA xpress system (GE Healthcare).

Tag-free fusion proteins were purified using a Praesto-HSA column followed by gel filtration (Superdex™ 200 HiLoad 26/600 or Superdex™ 75 HiLoad 26/600). Following SDS-PAGE analysis positive fractions were pooled and their protein concentrations were determined by absorbance measurement at 280 nm using the specific molar absorbent coefficient.

Further analysis included reversed phase chromatography (RP-HPLC) and analytic size exclusion chromatography (SE-HPLC). RP-HPLC was performed using an Ultimate 3000 HPLC system (Thermo Fisher Scientific) and a PLRP-S (5 μm, 300 Å) column (Agilent). SE-HPLC was performed using an Ultimate 3000 HPLC system (Thermo Fisher Scientific) and a Superdex 75 or Superdex 200 increase 5/150 GL (Cytiva). No aggregation was detected.

### Example 2. Functional Characterization: Specific Binding of the Fusion Proteins to Serum Albumin and to Targets (Surface Plasmon Resonance, SPR)

Recombinant protein A, HSA, MSA or target (either ED-B or Her2) was immobilized on a High Capacity Amine sensor chip (Bruker) after NHS/EDC activation with a Sierra SPR-32 system (Bruker). The chip was equilibrated with SPR running buffer (PBS 0.05%, Tween pH 7.3). Injection of ethanolamine after target immobilization was used to block free NHS groups. To determine the binding of fusion proteins to FAP, the Fc-tagged target was injected with 60 or 30 nM to Protein A coupled spots followed by the injection of fusion proteins.

Binding proteins were applied to the chip in serial dilutions with a flow rate of 30 μl/min. Upon binding, analyte accumulated on the surface increasing the refractive index. This change in the refractive index was measured in real time and plotted as response or resonance units versus time. The association was performed for 120 seconds and the dissociation for 180 seconds. After each run, the chip surface was regenerated with 30 μl regeneration buffer (10 mM glycine pH 2.0 or pH 1.5) and equilibrated with running buffer. Binding studies were carried out using the Sierra SPR-32 system (Bruker); data evaluation was operated via the Sierra Analyser software, provided by the manufacturer, by the use of the Langmuir 1:1 model (RI=0). FIG. 1 shows the binding affinity of fusion proteins to HSA and the specific domain of a tumor target (FIG. 1A: ED-B, FIG. 1B: Her2, FIG. 1C: FAP).

All fusion proteins showed binding affinity to mouse serum albumin (MSA).

Fusion proteins with albumin binding domains of SEQ ID NO: 3, 4, 5, 6, 7, 8, 9, 10, 11, 13, 17, 20, 21, 22, 23, 24, 25, 26, 27, 28, 29, 30, 32 showed high specific binding affinities to MSA below 16 nM.

Fusion proteins with albumin binding domains of SEQ ID NO: 3, 4, 5, 6, 7, 8, 9, 10, 11, 17, 20, 23, 26, 27, 28, 29, 30, 32 showed high specific binding affinities to MSA below 10 nM. Fusion proteins with albumin binding domains of SEQ ID NO: 3, 4, 8, 9, 10, 11, 17, 20, 27, 28, 29, 30, 32 showed high specific binding affinities to MSA below 5 nM.

Fusion protein comprising a FAP specific binding protein (SEQ ID NO: 42) and an albumin binding domain of SEQ ID NO: 9 have an affinity to MSA of or below 1 nM (e.g. fusion proteins of SEQ ID NO: 54 and SEQ ID NO: 47).

Ubiquitin (wildtype) does not bind to Her2, EDB, or FAP.

Fusion proteins with albumin binding domains c42 (SEQ ID NO: 34), c45 (SEQ ID NO: 35), c49 (SEQ ID NO: 37), and c50 (SEQ ID NO: 38), showed low affinity binding of minimal 100 nM to HSA or MSA.

### Example 3. Functional Characterization: Specific Binding of the Fusion Proteins to Cell Surface Expressed hFAP (Flow Cytometry)

Flow cytometry was used to analyze the interaction of fusion proteins (and as control FAP specific targeting moieties without albumin binding domain) with cell surface-exposed hFAP. FAP overexpressing human embryonic kidney cell line HEK293, and empty vector control HEK293-pEntry cells were used. The anti-hFAP antibody (R&D Systems, MAB3715-100) with a concentration of 1 μg/ml in combination with anti-mouse-IgG-Alexa488 (Invitrogen, A10680) with a 1:1000 dilution was used as positive control for hFAP-expressing cells. Results are summarized in Table 1.

Cells were trypsinized and resuspended in medium containing FCS, washed and stained in pre-cooled FACS blocking buffer (3% FCS/PBS). A suspension with a cell concentration of 1×10⁶ cells/ml was prepared for cell staining and filled with 100 μl/well into a 96 well plate (Greiner) in triplicate for each cell line.

Fusion proteins were tested with a concentration of 1 μM, 100 nM, 10 nM, 1 nM or 0.1 nM on hFAP expressing HEK293-hFAP-cells. To exclude an unspecific binding, fusion proteins were also incubated on control cells HEK293-pEntry with the same concentrations. Comparable amounts of wildtype ubiquitin (clone 139090) were used as negative control. Supernatants were removed after 45 min, cells were washed in blocking buffer and 100 μl/well rabbit anti-StrepTag antibody (GenScript; A00626), 1:300 diluted in FACS blocking buffer were added. After removal of the primary antibody, goat anti-human-IgG-Alexa Fluor 488 antibody (Invitrogen; A4008) was applied in a 1:1000 dilution. Flow cytometry measurement was conducted on the Guava easyCyte 5HT device from Merck-Millipore at excitation wavelength 488 nm and emission wavelength 525/30 nm.

The FACS experiments show that all fusion proteins bind to hFAP expressed on cell surfaces of HEK293 cells. A strong binding was observed for all fusion proteins (Table 1; shown by +++). No fusion protein showed binding to control cells (HEK293-pEntry). Anti-hFAP-antibody shows a positive staining on hFAP-expressing cells.

TABLE 1
Binding of fusion proteins to the cell surface
expressed target FAP on HEK293-hFAP-cells

| | | SEQ ID NO. | SEQ ID NO: | |
|---|---|---|---|---|
| CID | HED | HED | targeting moiety | HEK293-hFAP |
| 218687 | d01 | 9 | 41 | +++ |
| 218691 | c01 | 4 | 41 | +++ |
| 218692 | a18 | 3 | 41 | +++ |
| 218699 | d01 | 9 | 42 | +++ |
| 218703 | c01 | 4 | 42 | +++ |
| 218704 | a18 | 3 | 42 | +++ |
| 218708 | d02 | 10 | 42 | +++ |
| 217832 | — | — | 41 | +++ |
| 217990 | — | — | 42 | +++ |
| Ubiquitin | — | — | 43 | − |

### Example 4: Binding Affinity of Fusion Proteins on Target-Expressing Cells

Her2 expressing Sk-Br-3-cells and HEK293-hFAP overexpressing cells were thawed, washed with medium containing FCS, subsequently washed with FACS blocking buffer (PBS/0.1% Sodium Azide/3% FCS) and 100 μl seeded in 96-well round bottom plates with a density of 1×10⁶ cells/ml. Dilution series from 10 UM to 8.6 fM (CID 217781, CID 217787) and a dilution series of 3 UM to 0.2 μM (CID 218466, CID 218467, 218512, CID 218513, CID 218514, CID 218515, CID 218516) were incubated on Her2-expressing Sk-Br-3-cells and dilution series from 1 μM to 0.07 μM of fusion proteins 218690 and 218702 were incubated on hFAP-overexpressing HEK293-cell line for 45 min at 4° C. After centrifugation the supernatants were removed and cells were washed with FACS blocking buffer and 100 μl/well rabbit anti-Strep-Tag antibody (GenScript; A00626), 1:300 diluted in FACS blocking buffer were added. After removal of the primary antibody goat anti-rabbit IgG Alexa Fluor 488 antibody (Invitrogen; A4008) was applied in a 1:1000 dilution in FACS blocking buffer. Flow cytometry measurement was conducted on the Guava easyCyte 5HT device from Merck-Millipore at excitation wavelength 488 nm and emission wavelength 525/30 nm. Results are shown in Table 2. The * in table 2 refers to a fusion protein with the albumin binding domain as dimer (homo-dimer) and/or the targeting moiety as dimer.

TABLE 2
Binding affinity of fusion proteins on target expressing cells

| | | | SEQ ID | | |
|---|---|---|---|---|---|
| | | SEQ ID | targeting | K_{D} [nM] vs | |
| CID | HED | HED | moiety | target | cell line |
| 217781 | c19 | 23 | 40 | 2.05 | Sk-Br-3 |
| 217787 | c38 | 8 | 40 | 2.17 | Sk-Br-3 |
| 218466 | c20 | 6 | 40 | 2.69 | Sk-Br-3 |
| 218467 | a18 | 3 | 40 | 3.81 | Sk-Br-3 |
| 218512 | d01 | 9 | 40 | 1.72 | Sk-Br-3 |
| 218513 | d03 | 11 | 40 | 2.48 | Sk-Br-3 |
| 218514 | c24 | 7 | 40 | 2.19 | Sk-Br-3 |
| 218515 | c01 | 4 | 40 | 1.47 | Sk-Br-3 |
| 218516 | a17 | 2 | 40 | 2.57 | Sk-Br-3 |
| 206479 | — | — | 40 | 1.58 | Sk-Br-3 |
| 218690 | d01 | 9* | 41* | 0.3 | HEK293-hFAP |
| 218702 | d01 | 9* | 42* | 0.28 | HEK293-hFAP |

### Example 5. Stability of Fusion Proteins in Serum for a Prolonged Time (Determination of Binding Affinity to the Target by Flow Cytometry)

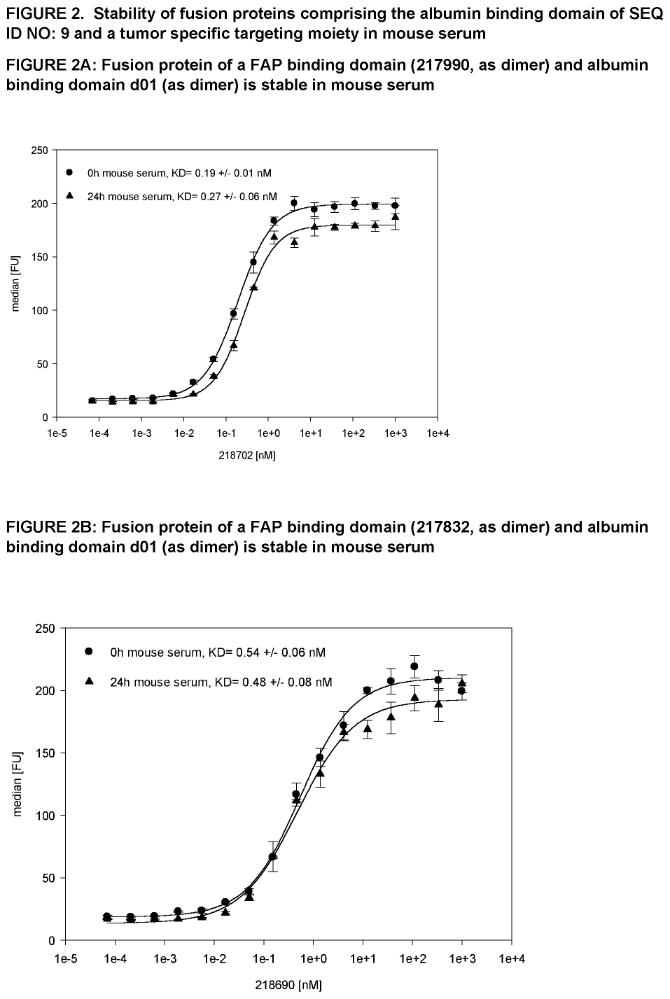

Dilution series from 1 μM to 0.07 μM of fusion protein 218690 and fusion protein 218702 were incubated in 100% mouse serum for 24 h at 37° C. hFAP expressing HEK293-cells were thawed, washed with medium containing FCS, subsequently washed with FACS blocking buffer (PBS/0.1% Sodium Azide/3% FCS) and 100 μl seeded in 96-well round bottom plates with a density of 1×10⁶ cells/ml. Dilution series of fusion proteins were incubated with human serum for 24 h and 0 h (control) at 37° C. HEK293-hFAP-cells were then incubated with dilution series for 45 min at 4° C. Cells were centrifuged and supernatants were removed. Cells were washed with FACS blocking buffer and 100 μl/well rabbit anti-Strep-Tag antibody (GenScript; A00626), 1:300 diluted in FACS blocking buffer were added. After removal of the primary antibody goat anti-rabbit IgG Alexa Fluor 488 antibody (Invitrogen; A4008) was applied in a 1:1000 dilution in FACS blocking buffer. Flow cytometry measurement was conducted on the Guava easyCyte 5HT device from Merck-Millipore at excitation wavelength 488 nm and emission wavelength 525/30 nm. Results are shown in FIG. 2. No significant difference in binding to hFAP was observed even after 24 h serum incubation. The fusion proteins are stable in mouse serum.

### Example 6. Stability of Fusion Proteins in Serum for a Prolonged Time (Determination of Binding Affinity to the Target by ELISA)

High binding plates (Greiner, 781061) were immobilized with 2.5 μg/ml hFAP-Fc or 1.2 μg/ml Her2-Fc over night at 4° C. Dilution series of 3 μM to 0.07 pM of fusion proteins with binding specifically to Her2 were incubated in 100% human serum or 100% mouse serum for 24 h at 37° C. Dilution series of 3 UM to 0.2 pM or 1 μM to 0.01 fM of fusion proteins with binding specifically to hFAP were incubated in 100% human serum or 100% mouse serum for 24 h at 37° C. ELISA-plates were washed three times with PBST (PBS+0.1% Tween) and blocked with 3% BSA/0.5% Tween/PBS up to 2 h at room temperature (rt). After preincubation for 0 h or 24 h in the presence of serum the dilution series were incubated on ELISA-plates for up to 1 h at rt. Wells were washed with PBST and incubated with biotinylated anti-ubiquitin-antibody (1:300) for up to 1 h at rt. The binding was visualized with Streptavidin-HRP (1:5.000).

Table 3 and Table 4 show the binding affinity to the respective target as K_{D} value after 0 h and 24 h incubation in serum. The fusion proteins comprise the albumin binding domain (referred to as HED) and the targeting moiety. The targeting moieties of SEQ ID NO: 41 and 42 bind to hFAP, respectively, and SEQ ID NO: 40 binds to Her2. * refers to a dimer; for example, 9* refers to SEQ ID NO: 9 as dimer in the fusion protein, 40* refers to SEQ ID NO: 40 as dimer in the fusion protein. There is no significant shift of K_{D} and no significant decreasing or increasing of the maximal binding was observed. The fusion proteins are stable in human serum and in mouse serum.

TABLE 3
Stability of fusion protein in mouse serum (ELISA)

| | | | | max | KD vs | KD vs | Increase |
|---|---|---|---|---|---|---|---|
| CID of | Albumin | SEQ ID | SEQ ID | binding | target 0 h | target 24 h | of KD vs |
| fusion | binding | alb. bind. | target. | after 24 | incubation | incuabtion | target |
| protein | domain | dom. | moiety | h [%] | [nM] | [nM] | [fold] |
| 218466 | c20 | 6 | 40 | 96.60 | 0.21 | 0.23 | 1.14 |
| 218467 | a18 | 3 | 40 | 104.20 | 0.17 | 0.20 | 1.20 |
| 218512 | d01 | 9 | 40 | 98.60 | 0.20 | 0.24 | 1.20 |
| 218513 | d03 | 11 | 40 | 106.50 | 0.25 | 0.31 | 1.23 |
| 218514 | c24 | 7 | 40 | 100.10 | 0.21 | 0.25 | 1.19 |
| 218515 | c01 | 4 | 40 | 107.60 | 0.18 | 0.21 | 1.16 |
| 218516 | a17 | 2 | 40 | 100.70 | 0.17 | 0.20 | 1.21 |
| 217781 | c19 | 23 | 40 | 97.71 | 0.36 | 0.63 | 1.72 |
| 217787 | c38 | 8 | 40 | 100.26 | 0.42 | 0.60 | 1.43 |
| 209467 | — | — | 40 | 102.20 | 0.16 | 0.12 | 0.77 |
| 218690 | d01 | 9* | 41* | 103.00 | 0.16 | 0.23 | 1.44 |
| 218700 | d01 | 9 | 42* | 103.39 | 0.10 | 0.12 | 1.20 |
| 218702 | d01 | 9* | 42* | 103.19 | 0.11 | 0.13 | 1.18 |
| 217832 | — | — | 41 | 106.98 | 0.31 | 0.71 | 2.29 |
| 217990 | — | — | 42 | 88.05 | 0.73 | 1.10 | 1.51 |

TABLE 4
Stability of fusion proteins in human serum (ELISA)

| | | SEQ | | | K_{D} vs | K_{D} vs | |
|---|---|---|---|---|---|---|---|
| | | ID | SEQ | max | target | target | increase |
| | Albumin | alb. | ID | binding | 0 h | 24 h | of K_{D} vs |
| | binding | bind. | target. | after 24 | incubation | incuabtion | target |
| CID | domain | dom. | moiety | h [%] | [nM] | [nM] | [fold] |
| 218466 | c20 | 6 | 40 | 95.12 | 0.34 | 0.32 | 0.93 |
| 218467 | a18 | 3 | 40 | 99.04 | 0.34 | 0.36 | 1.05 |
| 218512 | d01 | 9 | 40 | 100.29 | 0.23 | 0.36 | 1.58 |
| 218513 | d03 | 11 | 40 | 100.47 | 0.34 | 0.43 | 1.27 |
| 218514 | c24 | 7 | 40 | 96.37 | 0.29 | 0.30 | 1.06 |
| 218515 | c01 | 4 | 40 | 96.10 | 0.34 | 0.28 | 0.82 |
| 218516 | a17 | 2 | 40 | 98.87 | 0.39 | 0.28 | 0.71 |
| 217781 | c19 | 23 | 40 | 94.10 | 0.61 | 0.79 | 1.29 |
| 217787 | c38 | 8 | 40 | 96.24 | 0.84 | 0.53 | 0.63 |
| 209467 | — | — | 40 | 101.64 | 0.20 | 0.10 | 0.48 |
| 218690 | d01 | 9* | 41* | 107.52 | 0.95 | 1.33 | 1.40 |
| 218700 | d01 | 9 | 42* | 97.09 | 0.55 | 0.72 | 1.31 |
| 218702 | d01 | 9* | 42* | 103.96 | 1.05 | 1.00 | 0.95 |
| 217832 | — | — | 41 | 109.24 | 0.35 | 0.54 | 1.56 |
| 217990 | — | — | 42 | 93.75 | 1.17 | 1.18 | 1.01 |

### Example 7: Stability of Fusion Proteins in Serum for a Prolonged Time (Western Blot Analysis)

Fusion proteins comprising the ED-B-specific targeting moiety of SEQ ID NO: 39 and the albumin binding domain c45 (SEQ ID NO: 35; fusion protein 221202), c49 (SEQ ID NO: 37; fusion protein 221203), c50 (SEQ ID NO: 38; fusion protein 221204) or c46 (SEQ ID NO: 36; fusion protein 221248) were tested for stability in human serum for a long period (up to 2 days) of incubation. The fusion proteins were incubated in 100% human serum at 37° C. and fusion proteins 221248 and 221204 were incubated in 100% mouse serum at 37° C. with a concentration of 3.6 ng/μl for 48 h. After 0 h, 6 h, 24 h, and 48 h an amount of 30 ng was removed for analysis from the mixture. The protein-serum mixture was boiled at 95° C. for 5 min. 30 ng of fusion protein-serum mixture were applied on a stain-free gel (4-20% Mini-PROTEAN® TGX Stain-Free™ Protein Gels, #4568094, Bio-Rad). The proteins were transferred with the Gel Doc EZ System (#1708270, Bio-Rad) onto a PVDF-membrane (Trans-Blot Turbo Midi 0.2 μM PVDF Transfer Packs, #1704157, Bio-Rad) according to manufacturer's information. Membranes were blocked over night at 4° C. in blocking buffer (PBS, 0.1% Tween, 3% BSA). After 3 washing steps with PBST (PBS, 0.1% Tween), membranes were incubated with a biotinylated anti-ubiquitin-antibody (1:2000) in blocking buffer for 1 h at rt, followed by 3 washing steps and further incubation with StreptAvidin-HRP (#554066, BD Pharmingen, Becton Dickinson) at dilution of 1:5000 in blocking buffer. The proteins were visualized with Pierce ECL Western Blotting-Substrate (#32209, Thermo Fisher Scientific) according to manufacturer information. The blots showed that the fusion proteins are stable in human or mouse serum for the long time period of 48 h.

### Example 8: DOTA Coupling and Loading with Lu or in of Fusion Proteins 218462, 218464 and 220521

Fusion proteins comprising the Her2-specific targeting moiety of SEQ ID NO: 40 and the albumin binding domain c20 (SEQ ID NO: 6; fusion protein 218462) or d01 (SEQ ID NO: 9; fusion protein 218464) as well as fusion protein 220521 comprising the hFAP-specific targeting moiety (SEQ ID NO: 42 as dimer) and d01 (SEQ ID NO: 9, as dimer), fusion protein 220520 (SEQ ID NO: 53) comprising the hFAP-specific targeting moiety (SEQ ID NO: 42) and d01 (SEQ ID NO: 9, as dimer), fusion protein 220519 (SEQ ID NO: 54) comprising the hFAP-specific targeting moiety (SEQ ID NO: 42 as dimer) and d01 (SEQ ID NO: 9), or fusion protein 220518 (SEQ ID NO: 55) comprising the hFAP-specific targeting moiety of SEQ ID NO: 42 and d01 (SEQ ID NO: 9) were coupled with DOTA and loaded with Lu or In. Proteins were incubated with 20× excess of Maleimide-DOTA (Chematech) in 50 mM Hepes, 5 mM EDTA, 150 mM NaCl, pH 7.0 for 3 h at room temperature. Uncoupled Maleimide-DOTA was removed by Resource Q or Resource S followed by gel filtration (Superdex™ 75 HiLoad 16/600). To load DOTA with Lu or In proteins were incubated with equimolar concentration of indium (III) chloride or lutetium (III) chloride (Sigma) in 100 mM NaAc pH 5.5 at 50° C. for 60 min. After centrifugation proteins were desalted in PBS pH 7.3 and analyzed by MALDI-TOF, RP-HPLC, SE-HPLC and SPR.

Table 5: Binding affinities of Lu or in labelled fusion proteins to the target and to MSA

TABLE 5A
Her2 or FAP specific fusion proteins

| | 218462 | | 218462 | | 218464 | | 220521 | | 220521 | | 220518 | | 220519 | | 220520 | |
|---|---|---|---|---|---|---|---|---|---|---|---|---|---|---|---|---|
| | DOTA | | DOTA | | DOTA | | DOTA | | DOTA | | DOTA | | DOTA | | DOTA | |
| | In | | Lu | | Lu | | Lu | | In | | Lu | | Lu | | Lu | |
| K_{D(HER2)} | 5 | nM | 10 | nM | 6 | nM | | | | | | | | | | |
| K_{D(hFAP)} | | | | | | | <0.5 | nM | <0.5 | nM | 6 | nM | <1 | nM | 5.9 | nM |
| K_{D(MSA)} | 82 | nM | 108 | nM | 55 | nM | 0.9 | nM | 0.9 | nM | 38 | nM | 67 | nM | <1 | nM |

| | | | | | | | | | | | | | | | |
|---|---|---|---|---|---|---|---|---|---|---|---|---|---|---|---|
| Tm | 71° | C. | 70° | C. | 70° | C. | 64° | C. | 64° | C. | n.d. | 64° | C. | 66° | C. |

| | | | | | | | | |
|---|---|---|---|---|---|---|---|---|
| SE-HPLC | 99% | 99% | 99% | 95% | 94% | 100% | 94% | 100% |
| rp-HPLC | 97% | 95% | 98% | 97% | 97% | 92% | 94% | 94% |

TABLE 5B
ED-B specific fusion proteins

| | | | 219087 | | | | 219088 | |
|---|---|---|---|---|---|---|---|---|
| | 219087 | | DOTA | | 219088 | | DOTA | |
| | DOTA | | Lu | | DOTA | | Lu | |
| Alb. Bind. | d02 | | d02 | | c20 | | c20 | |
| domain | | | | | | | | |
| K_{D(ED-B)} | <1 | nM | <1 | nM | <1 | nM | <1 | nM |
| K_{D(HSA)} | 3.3 | nM | 1.3 | nM | 53.1 | nM | 39.5 | nM |
| K_{D(MSA)} | 1.7 | nM | 1.3 | nM | 62.5 | nM | 49.7 | nM |
| Tm | 66° | C. | 58° | C. | 67° | C. | 59° | C. |

| | | | | |
|---|---|---|---|---|
| rp-HPLC | 96% | 96% | 100% | 95% |

1. A fusion protein comprising
(a) a binding protein for serum albumin comprising the amino acid sequence of SEQ ID NO: 33, or an amino acid sequence with 1 or 2 substitutions, deletions, or insertions as compared to SEQ ID NO: 33, and having a binding affinity to serum albumin of less than 25 nM, and
(b) a targeting moiety having a binding affinity of less than 100 nM to a protein expressed by tumors.

2. The fusion protein according to claim 1, wherein the binding protein for serum albumin comprises an amino acid sequence as set forth in any of SEQ ID NOs: 1-32, or comprises an amino acid sequence with 1 or 2 substitutions, deletions, or insertions as compared to SEQ ID NOS: 1-32, and further wherein the fusion protein has a binding affinity to serum albumin of less than 25 nM.

3. The fusion protein according to claim 1 wherein the serum albumin is a human serum albumin or a mouse serum albumin.

4. The fusion protein according to claim 1, wherein the targeting moiety is a non-immunoglobulin protein, preferably a ubiquitin mutein, a mutein of domains of protein A, an ankyrin repeat protein mutein, a lipocalin mutein, a mutein of human Fyn SH3 domain, a mutein of the tenth domain of human fibronectin, a mutein of FN3 domain, a mutein of Kunitz domains, a Sac7d mutein, a chagasin mutein, a mutein of multimerized low density lipoprotein receptor-A, a mutein of cysteine-knot miniprotein, a mutein of Stefin, a mutein of Armadillo-repeat protein, a mutein of tetranectin, a mutein of C-type lectin domain, or a mutein of CTLA-4, or wherein the targeting moiety is an immunoglobulin, a fragment or variant thereof, a single domain antibody, or a single chain variable fragment (scFv) of an antibody.

5. The fusion protein according to claim 4, wherein the non-Immunoglobulin protein comprises an ubiquitin mutein exhibiting 80% to 94% identity to ubiquitin (SEQ ID NO: 43).

6. The fusion protein according to claim 1, wherein the targeting moiety binds to a protein expressed by tumors, optionally Her2, FAP, or ED-B.

7. The fusion protein according to claim 1, wherein the half-life of the targeting moiety of the fusion protein in serum or in blood is longer than the half-life of the targeting moiety without the serum albumin binding protein in serum or in blood.

8. A nucleic acid molecule encoding the fusion protein according to claim 1.

9. A vector comprising the nucleic acid molecule of claim 8.

10. A host cell or a non-human host comprising the fusion protein of claim 1.

11. A composition comprising the fusion protein of claim 1.

12. A method producing the fusion protein of claim 1, comprising culturing the host cell of claim 10 under conditions suitable to obtain said fusion protein, and optionally isolating said fusion protein.

13. A host cell or a non-human host comprising nucleic acid molecule of claim 8.

14. A host cell or a non-human host comprising the vector of claim 9.


SEQUENCE LISTING (55 sequences)

SEQ ID NO: 1 - protein, 44 aa, synthetic construct
  REGION 1..44: note = albumin binding protein a01
LAEAKVLAIKELDKAGISDYYFNLINNAKTVEGVKALKAQIVAA

SEQ ID NO: 2 - protein, 44 aa, synthetic construct
  REGION 1..44: note = albumin binding protein a17
LAEAKVLAIKELDKAGISNYYFNLINNAKTVEGVKALKAQIVAA

SEQ ID NO: 3 - protein, 44 aa, synthetic construct
  REGION 1..44: note = albumin binding protein a18
LAEAKVLAIKELDKAGISNYYFNLINNAKTVEGVKDLKAQIVAA

SEQ ID NO: 4 - protein, 44 aa, synthetic construct
  REGION 1..44: note = albumin binding protein c01
LAEAKVLALKELDKYGISNYYKNLINNAKTIEGVKALKAQIVAA

SEQ ID NO: 5 - protein, 44 aa, synthetic construct
  REGION 1..44: note = albumin binding protein c18
LAEAKVLALKELDKYGISNYYKNLINNAKTIEGVKALKDAIVAA

SEQ ID NO: 6 - protein, 44 aa, synthetic construct
  REGION 1..44: note = albumin binding protein c20
LAEAKVLALKELDKYGISNYYKNLINNAKTIEGVKALKDEIVAA

SEQ ID NO: 7 - protein, 44 aa, synthetic construct
  REGION 1..44: note = albumin binding protein c24
LAEAKVLALKELDKYGISNYYKNLINNAKTIEGVEALKDAILAA

SEQ ID NO: 8 - protein, 44 aa, synthetic construct
  REGION 1..44: note = albumin binding protein c38
LAEAKVLALKELDKYGISDFYKRLIDKAKTIEGVEALKDAILAA

SEQ ID NO: 9 - protein, 44 aa, synthetic construct
  REGION 1..44: note = albumin binding protein d01
LAEAKVLALKELDKYGISNYYKNLINNAKTVEGVKALKEAIVAA

SEQ ID NO: 10 - protein, 46 aa, synthetic construct
  REGION 1..46: note = albumin binding protein d02
LAEAKVLALKELDKYGISNYYKNLINNAKTVEGVKALKEAIVAALP

SEQ ID NO: 11 - protein, 44 aa, synthetic construct
  REGION 1..44: note = albumin binding protein d03
LAEAKVLALKELDKYGISNYYKNLINNAKTVEGVEALKEAILAA

SEQ ID NO: 12 - protein, 44 aa, synthetic construct
  REGION 1..44: note = albumin binding protein a03
LAEAKVLAIKELDKAGISDYYFNLINNAKTVDGVKALKAQIVAA

SEQ ID NO: 13 - protein, 44 aa, synthetic construct
  REGION 1..44: note = albumin binding protein a05
LAEAKVLAIKELDKAGISDYYFNLINNAKTIEGVKALKAQIVAA

SEQ ID NO: 14 - protein, 44 aa, synthetic construct
  REGION 1..44: note = albumin binding protein a21
LAEAKVLAIKELDKAGISNYYFNLINNAKTIEGVKALKAQIVAA

SEQ ID NO: 15 - protein, 44 aa, synthetic construct
  REGION 1..44: note = albumin binding protein a34
LAEAKVLAIKELDKAGISNYYFNLINNAKTVEGVKALKAQIVAR

SEQ ID NO: 16 - protein, 44 aa, synthetic construct
  REGION 1..44: note = albumin binding protein b01
LAEAKVLALKELDKYGVSDYYKNLINNAKTVEGVKALKAQIVAA

SEQ ID NO: 17 - protein, 44 aa, synthetic construct; identical to SEQ ID NO: 4
  REGION 1..44: note = albumin binding protein b53

SEQ ID NO: 18 - protein, 44 aa, synthetic construct
  REGION 1..44: note = albumin binding protein c08
LAEAKVLALKELDKYGISNYYKNLINNAKTIEGVEALKAQIVAA

SEQ ID NO: 19 - protein, 44 aa, synthetic construct
  REGION 1..44: note = albumin binding protein c10
LAEAKVLALKELDKYGISNYYKNLINNAKTIEGVKALKDQIVAA

SEQ ID NO: 20 - protein, 44 aa, synthetic construct
  REGION 1..44: note = albumin binding protein c11
LAEAKVLALKELDKYGISNYYKNLINNAKTIEGVKALKEQIVAA

SEQ ID NO: 21 - protein, 44 aa, synthetic construct
  REGION 1..44: note = albumin binding protein c12
LAEAKVLALKELDKYGISNYYKNLINNAKTIEGVKALKAAIVAA

SEQ ID NO: 22 - protein, 44 aa, synthetic construct
  REGION 1..44: note = albumin binding protein c14
LAEAKVLALKELDKYGISNYYKNLINNAKTIEGVKALKAQILAA

SEQ ID NO: 23 - protein, 44 aa, synthetic construct
  REGION 1..44: note = albumin binding protein c19
LAEAKVLALKELDKYGISNYYKNLINNAKTIEGVKALKEAIVAA

SEQ ID NO: 24 - protein, 44 aa, synthetic construct
  REGION 1..44: note = albumin binding protein c22
LAEAKVLALKELDKYGISNYYKNLINNAKTIEGVEALKDAIVAA

SEQ ID NO: 25 - protein, 44 aa, synthetic construct
  REGION 1..44: note = albumin binding protein c23
LAEAKVLALKELDKYGISNYYKNLINNAKTIEGVEALKEAIVAA

SEQ ID NO: 26 - protein, 44 aa, synthetic construct
  REGION 1..44: note = albumin binding protein c25
LAEAKVLALKELDKYGISNYYKNLINNAKTIEGVEALKEAILAA

SEQ ID NO: 27 - protein, 44 aa, synthetic construct
  REGION 1..44: note = albumin binding protein c29
LAEAKVLALKELDKYGISNYYKNLIDKAKTIEGVEALKDAILAA

SEQ ID NO: 28 - protein, 44 aa, synthetic construct
  REGION 1..44: note = albumin binding protein c37
LAEAKVLALKELDKYGVSNFYKRLIDKAKTIEGVEALKDAILAA

SEQ ID NO: 29 - protein, 44 aa, synthetic construct
  REGION 1..44: note = albumin binding protein c40
LAEAKVLALKELDKYGVSDFYKRLIDKAKTVEGVEALKDAILAA

SEQ ID NO: 30 - protein, 44 aa, synthetic construct
  REGION 1..44: note = albumin binding protein c44
LAEAKVLALKELDKYGISNYYKNLINRAKTIEGVKALKDEIVAA

SEQ ID NO: 31 - protein, 44 aa, synthetic construct
  REGION 1..44: note = albumin binding protein c47
LAEAKVLALKELDKYGISNYYKNLINNAKTIEGVEGLKDAILAA

SEQ ID NO: 32 - protein, 44 aa, synthetic construct; identical to SEQ ID NO: 27
  REGION 1..44: note = albumin binding protein c48

SEQ ID NO: 33 - protein, 44 aa, synthetic construct
  REGION 1..44: note = albumin binding domain
  VARIANT 9: note = Xaa may be selected from L or I
  VARIANT 15: note = Xaa may be selected from Y or A
  VARIANT 17: note = Xaa may be selected from I or V
  VARIANT 19: note = Xaa may be selected from N or D
  VARIANT 20: note = Xaa may be selected from an aromatic amino acid (Y or F)
  VARIANT 22: note = Xaa may be selected from K or F
  VARIANT 23: note = Xaa may be selected from N or R
  VARIANT 26: note = Xaa may be selected from N or D
  VARIANT 27: note = Xaa may be selected from N, K, or R
  VARIANT 31: note = Xaa may be selected from I or V
  VARIANT 32: note = Xaa may be selected from E or D
  VARIANT 35: note = Xaa may be selected from K or E
  VARIANT 39: note = Xaa may be selected from D, A, or E
  VARIANT 40: note = Xaa may be selected from E or Q
  VARIANT 42: note = Xaa may be selected from V or L
  VARIANT 44: note = Xaa may be selected from A or R
LAEAKVLAXKELDKXGXSXXYXXLIXXAKTXXGVXALKXXIXAX

SEQ ID NO: 34 - protein, 44 aa, synthetic construct
  REGION 1..44: note = albumin binding protein c42
LAEAKVLALKELDKYGISNYYKNLINNAKTIESVKALKDEIVAA

SEQ ID NO: 35 - protein, 44 aa, synthetic construct
  REGION 1..44: note = albumin binding protein c45
LAEAKVLALKELDKYGISNGYKNLINNAKTIEGVKALKDEIVAA

SEQ ID NO: 36 - protein, 44 aa, synthetic construct
  REGION 1..44: note = albumin binding protein c46
LAEAKVLALKELDKYGISNLYKNLINNAKTIEGVKALKDEIVAA

SEQ ID NO: 37 - protein, 44 aa, synthetic construct
  REGION 1..44: note = albumin binding protein c49
LAEAKVLALKELDKYGISNDYKNLINNAKTIEGVEALKDAILAA

SEQ ID NO: 38 - protein, 44 aa, synthetic construct
  REGION 1..44: note = albumin binding protein c50
LAEAKVLALKELDKYGISNYGKNLINNAKTIEGVEALKDAILAA

SEQ ID NO: 39 - protein, 155 aa, synthetic construct
  REGION 1..155: note = specific extradomain B (ED-B) binding ubiquitin mutein
MRIWVHTLTGKTITLEVEPSDTIENVKAKIQDKEGIPPDQQRLIWAGKQLEDGRTLSDYNINPKLSLHLVLRLRAAGIGMQIFVHTQTGKTITLEVEPSDTIENVKAKIQDKEGIPPDQQRLIWAGKQLEDGRTLSDYNIGWQAPLHLVLRLRAA

SEQ ID NO: 40 - protein, 152 aa, synthetic construct
  REGION 1..152: note = Her2 specific binding protein (ubiquitin mutein; 206479)
MQIFVKTLTGKTITLEVEPSDTTENVKAKIQDKEGIPPDQQTLAFVGKQLEDGRTLSDYNIQKESTLWLYLTLRAAMRIFVKTHTGKTITLDVEPSDTIENVKAKIQDKEGIPPDQQRLIWAGKQLEDGRTLSDYNIQEWAILHLVLRLRAA

SEQ ID NO: 41 - protein, 74 aa, synthetic construct
  REGION 1..74: note = FAP specific binding protein (217832)
MQIFVWTRINKLITLEVEPSDTIENVKAKIQDKEGIPPDQQSLYWKGKQLEDGRTLSDYNINIWPNLYLSLRAA

SEQ ID NO: 42 - protein, 82 aa, synthetic construct
  REGION 1..82: note = FAP specific binding protein 217990
MQIFVKTLTPEWHIIGKTITLEVEPSDTIENVKAKIQDKEGIPPDQQNLAWAGKQLEDGRTLSDYNIQGGSMLMLKLHLRAA

SEQ ID NO: 43 - protein, 76 aa, synthetic construct
  REGION 1..76: note = ubiquitin
MQIFVKTLTGKTITLEVEPSDTIENVKAKIQDKEGIPPDQQRLIWAGKQLEDGRTLSDYNIQKESTLHLVLRLRAA

SEQ ID NO: 44 - protein, 74 aa, synthetic construct
  REGION 1..74: note = FAP binding protein
MQIFVWTDDWKRITLEVEPSDTIENVKAKIQDKEGIPPDQQVLAWRGKQLEDGRTLSDYNIMVWPNLWLRLRAA

SEQ ID NO: 45 - protein, 217 aa, synthetic construct
  REGION 1..217: note = fusion protein 218462 (her2 binding protein 206479 x c20)
MQIFVKTLTGKTITLEVEPSDTTENVKAKIQDKEGIPPDQQTLAFVGKQLEDGRTLSDYNIQKESTLWLYLTLRAAMRIFVKTHTGKTITLDVEPSDTIENVKAKIQDKEGIPPDQQRLIWAGKQLEDGRTLSDYNIQEWAILHLVLRLRAAGGGGSGGGGSGGGGSGGGGSLAEAKVLALKELDKYGISNYYKNLINNAKTIEGVKALKDEIVAAC

SEQ ID NO: 46 - protein, 217 aa, synthetic construct
  REGION 1..217: note = fusion protein 218464 (her2 binding protein 206479 x d1)
MQIFVKTLTGKTITLEVEPSDTTENVKAKIQDKEGIPPDQQTLAFVGKQLEDGRTLSDYNIQKESTLWLYLTLRAAMRIFVKTHTGKTITLDVEPSDTIENVKAKIQDKEGIPPDQQRLIWAGKQLEDGRTLSDYNIQEWAILHLVLRLRAAGGGGSGGGGSGGGGSGGGGSLAEAKVLALKELDKYGISNYYKNLINNAKTVEGVKALKEAIVAAC

SEQ ID NO: 47 - protein, 283 aa, synthetic construct
  REGION 1..283: note = fusion protein 220521 (dimer of FAP binding protein 217990 x dimer d01)
MQIFVKTLTPEWHIIGKTITLEVEPSDTIENVKAKIQDKEGIPPDQQNLAWAGKQLEDGRTLSDYNIQGGSMLMLKLHLRAAGGGGSGGGGSMQIFVKTLTPEWHIIGKTITLEVEPSDTIENVKAKIQDKEGIPPDQQNLAWAGKQLEDGRTLSDYNIQGGSMLMLKLHLRAAGGGGSGGGGSLAEAKVLALKELDKYGISNYYKNLINNAKTVEGVKALKEAIVAAGGGGSGGGGSLAEAKVLALKELDKYGISNYYKNLINNAKTVEGVKALKEAIVAAC

SEQ ID NO: 48 - protein, 10 aa, synthetic construct
  REGION 1..10: note = linker
GGGGSGGGGS

SEQ ID NO: 49 - protein, 20 aa, synthetic construct
  REGION 1..20: note = linker
GGGGSGGGGSGGGGSGGGGS

SEQ ID NO: 50 - protein, 15 aa, synthetic construct
  REGION 1..15: note = linker
GGGGSGGGGSGGGGS

SEQ ID NO: 51 - protein, 14 aa, synthetic construct
  REGION 1..14: note = amino acids 1-14
LAEAKVLALKELDK

SEQ ID NO: 52 - protein, 14 aa, synthetic construct
  REGION 1..14: note = N-terminal amino acids of serum albumin binding domain
LAEAKVLAIKELDK

SEQ ID NO: 53 - protein, 190 aa, synthetic construct
  source 1..190: note = FAP binding fusion protein 220520
MQIFVKTLTPEWHIIGKTITLEVEPSDTIENVKAKIQDKEGIPPDQQNLAWAGKQLEDGRTLSDYNIQGGSMLMLKLHLRAAGGGGSGGGGSLAEAKVLALKELDKYGISNYYKNLINNAKTVEGVKALKEAIVAAGGGGSGGGGSLAEAKVLALKELDKYGISNYYKNLINNAKTVEGVKALKEAIVAA

SEQ ID NO: 54 - protein, 229 aa, synthetic construct
  source 1..229: note = FAP binding fusion protein 220519
MQIFVKTLTPEWHIIGKTITLEVEPSDTIENVKAKIQDKEGIPPDQQNLAWAGKQLEDGRTLSDYNIQGGSMLMLKLHLRAAGGGGSGGGGSMQIFVKTLTPEWHIIGKTITLEVEPSDTIENVKAKIQDKEGIPPDQQNLAWAGKQLEDGRTLSDYNIQGGSMLMLKLHLRAAGGGGSGGGGSLAEAKVLALKELDKYGISNYYKNLINNAKTVEGVKALKEAIVAAC

SEQ ID NO: 55 - protein, 136 aa, synthetic construct
  source 1..136: note = FAP binding fusion protein 220518
MQIFVKTLTPEWHIIGKTITLEVEPSDTIENVKAKIQDKEGIPPDQQNLAWAGKQLEDGRTLSDYNIQGGSMLMLKLHLRAAGGGGSGGGGSLAEAKVLALKELDKYGISNYYKNLINNAKTVEGVKALKEAIVAA